(19) United States
(12) Patent Application Publication
(10) Pub. No.: US 2013/0101553 A1
(43) Pub. Date: Apr. 25, 2013

(54) INDUCING INACTIVATION OF FIBROGENIC MYOFIBROBLASTS

(71) Applicants: Tatiana KISSELEVA, La Jolla, CA (US); David BRENNER, La Jolla, CA (US)

(72) Inventors: Tatiana KISSELEVA, La Jolla, CA (US); David BRENNER, La Jolla, CA (US)

(73) Assignee: THE REGENTS OF THE UNIVERSITY OF CALIFORNIA, Oakland, CA (US)

(21) Appl. No.: 13/450,400

(22) Filed: Apr. 18, 2012

Related U.S. Application Data
(60) Provisional application No. 61/476,556, filed on Apr. 18, 2011.

(51) Int. Cl. A61K 31/513; A61K 31/395; A61K 31/426; A61K 31/215; A61K 31/216; A61K 31/192

(52) U.S. Cl. CPC A61K 31/513; A61K 31/216; A61K 31/192; A61K 31/426; A61K 31/215; A61K 31/395
USPC 424/85.5; 435/34; 514/543; 514/274; 514/571; 514/369; 514/530; 514/183

(57) ABSTRACT

Provided are methods of treating fibrotic conditions in a subject and diagnostic methods for determining fibrosis and appropriate treatments for the fibrosis by the identification of specific subsets of fibrogenic myofibroblasts.

### CROSS REFERENCE TO RELATED APPLICATIONS

This application claims the benefit of priority to U.S. Provisional Application Ser. No. 61/476,556, filed Apr. 18, 2011, which is hereby incorporated by reference in its entirety, including all figures.

### STATEMENT OF GOVERNMENT SUPPORT

This invention was made with government support under grant no. AA011999 awarded by the NIH. The government has certain rights in the invention.

### FIELD OF THE INVENTION

The present invention is in the field of therapeutics and diagnostics related to fibrosis in animals, preferably humans.

### BACKGROUND OF THE DISCLOSURE

Fibrogenic myofibroblasts are cells responsible for collagen production and making the tissues fibrotic, the process associated with tissue destruction in organs capable of developing fibrosis, such as heart, lung, liver, kidney and skin diseases. Chronic liver injury of any etiology produces fibrosis as a result of deregulation of the normal healing process with massive accumulation of extracellular matrix (ECM), including type I collagen (ColI)(1). Myofibroblasts are ColI⁺ α-smooth muscle actin (a-SMA)⁺ cells that produce the ECM scar in fibrosis. One of the most important concepts in clinical and experimental liver fibrosis is reversibility. Removal of the etiological source of the chronic injury in patients (e.g. HBV, HCV, biliary obstruction, or alcohol) and in rodents (CCl₄ or bile duct ligation) produces regression of liver fibrosis and is associated with decreased cytokine and ECM production, increased collagenase activity, and the disappearance of myofibroblasts(1, 2). During regression of fibrosis, some myofibroblasts undergo senescence(3) and apoptosis(2). However, the number of apoptotic myofibroblasts and the fate of the remaining myofibroblasts in the recovering liver is unknown.

Hepatic stellate cells (HSCs), the liver pericytes that store retinoids, are a major source of myofibroblasts in hepatotoxic liver fibrosis(4). Liver injury results in activation of quiescent HSCs (qHSCs), which proliferate and undergo phonotypical and morphological changes characteristic of myofibroblasts. Removal of the injurious agent results in the clearance of activated HSCs (aHSCs) by the cytotoxic action of natural killer cells (1), and is linked to upregulation of ligands of NK cell receptor NKG2D, MICA and ULBP2, in senescent aHSCs(3). Although never demonstrated in vivo, studies in culture suggest that aHSCs can revert to a more quiescent phenotype(5), characterized by expression of adipogenic genes and loss of fibrogenic gene expression(5).

### SUMMARY OF THE INVENTION

The disclosure provides therapeutic methods. An embodiment of the disclosure provides a method for reducing one or more symptoms of fibrosis of parenchymal organs, such as, without limitation, liver fibrosis, renal fibrosis, skin fibrosis, and/or pulmonary fibrosis in a subject by administering to a subject a therapeutic amount of a compound or compounds that upregulate an inactivation-associated gene product, for example, Hspa1a/b gene in an activated cell, such as a hepatic stellate cells (aHSC) to produce an inactivated cell, e.g., an inactivated hepatic stellate cell (iHSC).

Disclosed herein is a method for reducing one or more symptoms of fibrosis in a subject by administering to the subject a therapeutic amount of one or more compounds that upregulate one or more of Hspa1a/b gene, PPARα, PPARγ, HSP70, HSP40, Hyaluronan synthase 1, GATA2, C/EBPa, BMPS, septin 4, Bambi, cathepsin S and H, neural proteins: synaptogyrin 1, synaptotagmin XIII, GFAP, transcription factors: Spi-C transcription factor (spi/PU.1 related), Spi-B transcription factor (spi-1/PU.related), PU.1-IRF, IRF-1 and 3 and 5, ISRE, Stat1, Pax5, Mafk2, ISGF3-g1; BL34 regulator of G-protein signaling 1, Rnd1-Rho family GTPase, in an activated fibrogenic myofibroblast cell or fibrogenic myofibroblast-like cell in an amount sufficient to decrease or inhibit the fibrosis.

Compounds used in the method can be selected from a PPARα agonist, PPARγ agonist, Hsp70 upregulator, HSP40 upregulator, Hspa1a/b upregulator, Hyaluronan synthase 1 upregulator or GATA2 upregulator.

In an embodiment of the method, the compound or compounds administered upregulate PPARγ, PPARα and/or Hspa1a/b.

In some embodiments of the method, PPARα agonists, and/or PPARγ agonists are used in combination with one or more Hsp70 upregulator, HSP40 upregulator, Hspa1a/b upregulator, Hyaluronan synthase 1 upregulator or GATA2 upregulator.

In some embodiments of the method the PPARα agonist is fenofibrate, WY14643, gemfibrozil, or ciprofibrate.

In some embodiments, the PPARγ agonist is thiazolidinediones, or 15-deoxy-delta (12, 14)-prostaglandin J2.

In other embodiments, the HSP70 and HSP40 upregulator is 17-allyamino-demthoxygeldanamycin.

In still other embodiments, the Hspa1a/b upregulator is taurolidine or tumor necrosis factor receptor apoptosis inducing ligand.

The methods described herein can be used to treat a fibrotic condition such as a fibrotic condition of the lung, liver, heart, kidney, skin, gastrointestinal tract or a combination thereof.

In other embodiments, the method can be used to treat a fibrotic condition of the liver chosen from fatty liver disease, steatohepatitis, primary and secondary biliary cirrhosis, cirrhosis, alcohol induced liver fibrosis, biliary duct injury, biliary fibrosis, hepatic fibrosis associated with hepatitis infection, autoimmune hepatitis, non-alcoholic fatty liver disease or progressive massive fibrosis.

In an embodiment of the method, the compound or compounds induce inactivation of fibrogenic myofibroblast or fibrogenic myofibroblast-like cells. In an aspect of this embodiment, the fibrogenic myofibroblast-like cell is a hepatic stellate cell.

In another embodiment of the disclosure, the compound or compounds of the method are given in combination with other antifibrotics, corticosteroids, anti-inflammatories, immunosuppressants, chemotherapeutic agents, anti-metabolites, and/or immunomodulators.

In another embodiment of the disclosure, the compound or compounds of the method are given in combination with one or more of the following: adefovir dipivoxil, candesartan, colchicine, combined ATG, mycophenolate mofetil, and tacrolimus, combined cyclosporine microemulsion and tacrolimus, elastometry, everolimus, FG-3019, Fuzheng Huayu, GI262570, glycyrrhizin (monoammonium glycyrrhizinate, glycine, L-cysteine monohydrochloride, interferon gamma-1b, irbesartan, losartan, oltipraz, ORAL IMPACT®., peginterferon alfa-2a, combined peginterferon alfa-2a and ribavirin, peginterferon alfa-2b (SCH 54031), combined peginterferon alpha-2b and ribavirin, praziquantel, prazosin, raltegravir, ribavirin (REBETOL®., SCH 18908), ritonavir-boosted protease inhibitor, pentoxyphilline, tacrolimus, tauroursodeoxycholic acid, tocopherol, ursodiol, or warfarin.

The invention also provides diagnostic methods. In one embodiment, the invention provides a method for detecting myofibroblasts in a sample, for example, hepatic stellate cells (HSCs) by determining the presence of at least one myofibroblast marker, for example, detecting an HSC marker selected from vitamin A+, Collagen+, Desmin+, GFAP+, CD146+.

In another embodiment of the diagnostic method, the invention provides a method for detecting portal fibroblasts (PFs) in a sample by determining the presence of at least one PF marker selected from Vitamin A−, Collagen+, Thy1.1+, and Elastin+, Mesothelin+.

In still another embodiment of the diagnostic method, the invention provides a method for distinguishing portal fibroblasts (PFs) and Hepatic Stellate Cells (HSCs) in a sample by determining at least one of following:
a) the presence of at least one HSC marker selected from vitamin A+, Collagen+, Desmin+, GFAP+, CD146+, and
b) the presence of at least one PF marker selected from Vitamin A−, Collagen+, Thy1.1+, and Elastin+, Mesothelin+.

An embodiment of the diagnostic method is the utilization of flow cytometry.

The disclosure additionally provides in one embodiment a method for diagnosing liver fibrosis in a subject by determining at least one of the following
a) determining, in a liver sample that contains fibrogenic myofibroblasts from a subject, the presence of portal fibroblasts (PFs) and Hepatic Stellate Cells (HSCs), and
b) determining the level of at least one of said portal fibroblasts (PFs) and of said Hepatic Stellate Cells (HSCs) in a liver sample, and
c) determining the ratio of portal fibroblasts (PFs) to Hepatic Stellate Cells (HSCs) in the liver sample.

The method for diagnosing liver fibrosis can also include the step of determining the contribution of myofibroblasts of other origins, such as CD45+ Collagen + fibrocytes.

### DESCRIPTION OF THE DRAWINGS

FIG. 1 shows regression of liver fibrosis is accompanied by loss of myofibroblasts. A. A comparison of the livers of Col-GFP mice that were untreated, CCl₄-treated (2 mo.), or recovered from CCl₄ (1 mo and 4 mo) with respect to GFP expression, Sirius Red staining and α-SMA immunohistochemistry. Representative bright field and fluorescent micrographs are shown using ×10 and ×20 objectives. B. Quantification of same four groups in (A) with respect to hydroxyproline content, Sirius Red staining, α-SMA immunofluorescence, GFP expression, collagen-α1(I) mRNA level, and α-SMA mRNA level, *p<0.01, **p<0.05. C. HSCs (Vitamin A⁺) constitute >90% of myofibroblasts (Vitamin A⁺GFP⁺), as detected by flow cytometry of the non-parenchymal cell fraction from CCl₄-treated (2 mo) Col-GFP mice (n=3). D. CCl₄ induces activation of qHSCs into aHSCs/myofibroblasts. Cre-loxP based genetic labeling marks the fate of collagen Type I-expressing aHSCs/myofibroblasts (see FIG. 6). During recovery from CCl₄-liver fibrosis, aHSCs may 1) apoptose (no genetically labeled YFP⁺ HSCs will remain in the liver), 2) inactivate (all YFP⁺ cells survive) or 3) some apoptose and some inactivate (YFP⁺ iHSCs will number <100% of aHSCs).

FIG. 2 shows genetically labeled aHSCs persist after 1 mo recovery. A. Livers from Collagen-α2(I)^{Cre-YFP} mice (no injury n=4; CCl₄-treated n=8; recovered 1 mo n=10) were co-stained for YFP, GFAP, Desmin, α-SMA. Genetically labeled HSCs were identified after 1 mo. recovery by YFP⁺ expression in Desmin⁺ or GFAP⁺ cells. The number of YFP⁺ HSCs is calculated relative to total HSCs (100%, merge 1, p<0.05 comparing CCl₄ and recovery groups). Nuclei are shown (DAPI, merge 2). B. HSCs (Vitamin A⁺) from Collagen-α2(I)^{Cre-YFP} mice (no injury n=4; CCl₄-treated n=6; recovered 1 mo n=6) were analyzed by flow cytometry. Genetically labeled aHSC and iHSCs were identified by simultaneous Vitamin A⁺ and YFP⁺ expression. Dot plots are shown, p<0.01 (comparing YFP⁺ aHSC and YFP⁺ iHSCs). C. Genetically labeled GFP⁺ HSCs persist in the livers of tamoxifen-inducible Col-α2(1)^{ER-Cre-GFP} after 1 mo. of recovery from CCl₄. To avoid genetic labeling of HSCs during development, tamoxifen-inducible Col-α2(1)^{ER-Cre-GFP} mice were generated by crossing Col-α2(1)^{ER-Cre} mice×Rosa26^{flox-Stop-mTRed-flox-mGFP} mice (here labeled as Rosa26^{f/f-mTRed} mice), treated with CCl₄ (2 mo), and genetic pulse-labeling of aHSCs was induced by daily tamoxifen administration during the last week of CCl₄ treatment, following by 1 month of reversal of liver fibrosis. Genetically labeled HSCs were visualized by immunostaining for membrane-tagged GFP⁺ (and simultaneous loss of mTRed⁻ expression, merge 1), DAPI-stained nuclei (merge 2), are taken with ×20 objective. The number of genetically labeled GFP⁺ HSCs is calculated as percent of Desmin⁺ HSCs (100%, merge 3). Genetic labeling of 35±6% aHSCs was achieved in response to CCl₄. 14±4% of GFP⁺ iHSCs persisted in the liver after 1 mo recovery (p<0.05, CCl₄ and recovery groups are compared), confirming that CCl₄-activated HSCs (and their progeny) remain in the liver after regression of fibrosis.

FIG. 3 shows HSCs (1 mo recovery) acquire a new phenotype distinct from qHSCs. A. HSCs from Col-α1(1)^{Cre-YFP} mice, uninjured or after 1 mo. recovery, were cultured for 48 h±TGF-β1 (2 ng/ml, for 6 h), and analyzed by RT-PCR for expression of fibrogenic and neural genes, *p<0.01, **p<0.05. B. CCl₄-treated Col-GFP mice (2×CCl₄; n=4) recuperated for 6 mo., then subjected to recurrent CCl₄-injury. Development of liver fibrosis in these mice was compared to littermates treated with CCl₄ only the second time (1×CCl₄, n=4) by Sirius Red. The number of aHSCs was estimated by fluorescent microscopy for Desmin and α-SMA (p<0.05, using ×20 objective). Total collagen deposition was measured by Hydroxyproline assay, *p<0.01. C. HSCs were isolated from Collagen-α1(I)-GFP/β-actin-RFP mice, uninjured or after recovery (7 days or 1 mo) from CCl₄ injury, and transferred intrahepatically (2.2×10⁵ cells) into 1 day old Rag2^{-/-}γc^{-/-} pups. Following CCl₄-injury, the number of RFP⁺GFP⁺ engrafted qHSCs, HSCs after 7 days and 1 mo. was calculated relative to number of total HSCs (detected by Desmin).

FIG. 4 shows genetically labeled HSCs obtain a new “inactivated” phenotype after 1 mo. of recovery. A. Microarray analysis: Vitamin A⁺ HSCs were sort purified from Col-α2(I)^{Cre-YFP} mice that were untreated (n=6), fibrotic (n=6), 7 days of recovery (n=3), and 1 mo of recovery (n=6). YFP⁺ and YFP⁻ HSCs were then subjected to the whole mouse genome microarray. Representative cell number is shown for each HSC group. B. YFP⁺ iHSCs (1 mo recovery) downregulate mRNAs of fibrogenic genes, and upregulate PPARγ, Bambi but not other “quiescent” HSC genes (Adfp, Adipor1, GFAP). The results are relative mRNA level (average of normalized values/multiple probes/gene) obtained using Agilant microarray, *p<0.01, **p<0.001. C. Gene expression profile clustering analysis identifies similarity between the different HSC phenotypes. The correlation coefficient was used to compare the qHSCs (1.00) gene expression pattern with YFP⁺ iHSCs (0.76), and aHSCs (0.63) expression patterns. D. Expression of signature genes was determined for YFP⁺ iHSCs (1 mo) and YFP⁺ HSCs (7 days recovery, 7 d), and fold induction (compared to YFP⁺ aHSCs) is shown for each group.

FIG. 5 shows genetically labeled YFP⁺ HSCs upregulate pro-survival Hsp1a/b genes at 7 days of recovery. A. Upregulation of pro-survival Hsp1a/b genes in YFP⁺ HSCs at 7 days of recovery. The results are expressed as relative mRNA levels (average of normalized values/multiple probes/gene, *p<0.001) obtained by Agilant microarray. B. Apoptosis was induced in Hspa1a/b^{-/-} and wild type HSCs by glyotoxin (25 nM for 4 h) and TNF-α (20 ng/ml)+Actinomycin (0.2 μg/ml) for 18 h. Cell morphology (BF), Vitamin A and apoptitic cells (TUNEL⁺ staining) are shown using ×10 objective. C. Hspa1a/b^{-/-} and wt mice were gradually subjected to CCl₄-injury and recovered for 2 weeks, livers were analyzed by Sirius Red, staining for Desmin and α-SMA (positive areas are indicated). Regression of fibrosis and disappearance of fibrogenic myofibroblasts during recovery were calculated in comparison with CCl₄-treatment (100%) and shown as percent of Sirius Red, Desmin and α-SMA positive areas, *p, 0.01, **p<0.05.

FIG. 6 shows HSCs are the major source of activated myofibroblasts in response to CCl₄-induced liver injury. A nonparenchymal fraction was isolated from CCl₄-treated Col-GFP mice. Activated myofibroblasts were identified by Col-GFP expression and sort purified. GFP+myofibroblasts were then sort purified into two fractions: Vitamin A⁺ and Vitamin A⁻. Vitamin A⁺GFP⁺ and Vitamin A⁻GFP⁺ myfibroblasts were plated and cell composition was analyzed. Expression of Vitamin A was confirmed by fluorescent microscopy. Phenotyping of GFP⁺ myofibroblasts by immunocytochemistry confirmed that >95% of Vitamin A⁺GFP⁺ express markers of HSCs (GFAP, Desmin), while >90% of Vitamin A⁻GFP⁺ express markers of portal fibroblasts. aHSCs (Vitamin A⁺) constitute >90% of myofibroblasts (Vitamin A⁺GFP⁺), as detected by flow cytometry of the non-parenchymal cell fraction from CCl₄-treated (2 mo) Col-GFP mice (n=3).

FIG. 7 shows genetic labeling of aHSCs in Col-α2(I)^{YFP} mice. Col-α2(I)^{YFP} mice were generated by crossing Collagen-α2(I)^{Cre} mice with Rosa26^{f/f-YFP} mice. Upon activation of collagen promoter (during development or in response to CCl₄) Cre-LoxP recombination occurred and resulted in permanent labeling of aHSCs and their progeny by YFP expression.

FIG. 8 shows some activated HSCs apoptose during recovery from liver fibrosis. A. Apoptotic TUNEL⁺ (594 nm, Roche) cells were detected at the highest level after 7 days of recovery from CCl₄. Fluorescent micrographs are visualized using ×10 objective and ×40 objectives. B. Apoptotic aHSCs were identified by co-localization of immunostaining for cleaved caspase-3 in GFP⁺ myofibroblasts in livers from Col-GFP mice, after 7 days of recovery from CCl₄ compared to uninjured mice (p<0.05). Fluorescent micrographs are shown using ×10, 20 and 40 objectives.

FIG. 9 shows genetically labeled YFP⁺Desmin⁺SMA⁻ HSCs persist in livers of Collagen-α1(I)^{Cre-YFP} mice after 1 mo recovery from CCl₄. A. Generation of collagen-α1(I)^{Cre} mice. Transgenic construct consists of collagen-α1(I) enhancer (1.7 kb) and promoter (3.2 kb), Cre gene (1 kb) and polyA site (0.3 kb). PCR of the genomic DNA has identified three founders. Founder N3 was used for this study. B. Livers from Collagen-α1(I)^{Cre-YFP} mice (no injury n=4; CCl₄-treated n=10; recovered 1 mo n=10) were co-stained for YFP, GFAP, Desmin and α-SMA. Genetically labeled HSCs were identified after 1 mo recovery by YFP⁺ expression in Desmin⁺ or GFAP⁺ cells. The number of YFP⁺ HSCs is relative to total HSCs (100%, in yellow). p<0.04 (comparing CCl₄ and recovery groups). C. Livers from Col-α1(I)^{Cre-YFP} mice were co-stained for YFP and Desmin (or α-SMA), and analyzed by confocal microscopy using ×60 objective. Genetically labeled inactivated HSCs were identified after 1 mo. recovery by YFP⁺ expression in SMA⁻Desmin⁺ cells. D. Livers from Col-α1(I)^{Cre-YFP} mice were co-stained for YFP and α-SMA, and analyzed by fluorescent microscopy using ×20 objective. Genetically labeled inactivated HSCs were identified after 4 mo. recovery as YFP⁺SMA⁻ cells. E. Vitamin A⁺ HSCs from Col-α1(I)^{Cre-YFP} mice 1 (no injury n=3; CCl₄-treated n=3; recovered 1 mo. n=3) were analyzed by flow cytometry. Genetically labeled HSC were identified by Vitamin A⁺ and YFP⁺ expression. Dot plots are shown, p<0.01 (comparing YFP⁺ aHSC and YFP⁺ iHSCs). F. Genetically labeled inactivated HSCs locate to the peri-sinusoidal space of Disse after 1 month recovery from CCl₄-induced fibrosis. Immunostaining for YFP and PECAM-1 was performed on formalin-fixed livers from Col-α1(I)^{Cre-YFP} mice and analyzed using ×20 objective. Nuclei are visualized by DAPI. Immunohistochemistry was performed on formalin-fixed livers from Col-α1(I)^{Cre-YFP} mice and analyzed using ×20 objective. YFP⁺ cells are visualized by staining with anti-GFP Ab and DAB, and counterstaining with Hematoxilin.

FIG. 10 shows some quiescent HSCs have a history of collagen expression during development. Quiescent HSCs were isolated from livers of Col-α2(I)^{Cre-FP} mice (8 weeks old, n=3) and analyzed by flow cytometry. Genetically labeled Vitamin A⁺YFP⁺ HSCs (20%) were detected in HSC fraction (100%), and exhibited larger size and higher granularity, as demonstrated using forward (FSC-A) and side (SSC-A) scatter. Representative dot plots are shown.

FIG. 11 shows that some HSCs transiently express collagen Type I during development. A. Livers from Col-GFP mice were obtained at day 16.5 of embryonic development (E16.5, n=5), and postnatally at day 1 (P1, n=4) and day 14 (P14, n=5), and compared to livers from adults (8 weeks old, uninjured or CCl₄-treated). GFP⁺ cells were detected in livers of E16.5-P14 mice (but not in uninjured adult mice), and were scattered throughout the acini (fluorescent micrographs, ×10 objective). In contrast to CCl₄-treated adult mice, the Col-GFP mice at E16.5-P14 did not have fibrosis (Sirius Red staining, bright field micrographs, taken using ×10 objective). The number GFP⁺ cells were minimal (or absent) at E12.5 and P25 (not shown). CV-central vein. B. Livers from Col-GFP mice at P14 were stained for HSC markers. 46±8% of all Desmin⁺ (100%) HSCs/myofibroblasts expressed GFP. Similarly, 49±6% of GFAP⁺ HSCs/myofibroblasts expressed GFP⁺. Expression of α-SMA was only detected around blood vessels in 3±1.5% of GFP⁺ cells. Fluorescent micrographs, images are taken using ×20 and ×60 objectives. C. HSCs were isolated from livers of Col-GFP mice using cell sorting for Vitamin A⁺ and GFP⁺ cells (n=4). Expression of GFP was detected in 38±4% of Vitamin A⁺ cells. Representative dot plots are shown. Sort purified Vitamin⁺GFP⁺ cells were plated for 18 h and stained for HSC markers. Vitamin⁺GFP⁺ cells expressed Desmin and GFAP. Fluorescent micrographs, ×40 objective. D. Microarray analysis: Vitamin A⁺GFP⁺ HSCs (P14) were sort purified from Col-GFP mice, and their gene expression profile was analyzed by the whole mouse genome microarray. Vit.A⁺GFP⁺ qHSCs (P14) were compared to 1) qHSCs with a history of collagen expression (YFP⁺, n=3) from uninjured Collagen-α2(I)^{Cre-YFP} mice; 2) qHSCs with no history of collagen expression (YFP⁻, n=3); and 3) aHSCs (YFP⁺, n=6) from CCl₄-treated Collagen-α2(I)^{Cre-YFP} mice. HSCs (P14) expressed collagen Type I and α-SMA at levels higher than in qHSCs but lower than in aHSCs. The results are relative mRNA level (average of normalized values/multiple probes/gene) obtained by Agilant microarray, *p<0.01, **p<0.001. E. Venn diagrams of the cell group-enriched genes that exhibited >2 fold up-regulation as compared to other groups. Vit.A⁺GFP⁺ HSCs (P14) are compared to YFP⁻ qHSCs and YFP⁺ aHSCs (left diagram). In addition, YFP⁻ and YFP⁺ qHSCs are compared to each other and to YFP⁺ aHSCs. The numbers of genes without group-specific expression are shown in the middle areas. F. Expression of signature genes (upregulated or downregulated) in Vit.A⁺GFP⁺ HSCs (P14) was determined in comparison with the average value of gene expression in qHSCs and aHSCs, and fold induction is shown for each group.

FIG. 12 shows GFP⁺ HSCs persist in the liver of Col-α2(1)^{ER-Cre-GFP} after 1 mo of recovery from CCl₄. A. Livers Col-α1(I)^{ER-Cre-GFP} mice were stained with anti-GFP antibody. Fluorescent micrographs, images are taken using ×10 objective. B. Livers from Col-α1(I)^{ER-Cre-GFP} mice were stained for Desmin or α-SMA and analyzed by confocal microscopy using ×60 objective using pseudocolors, ns—non-specific. Genetically labeled inactivated HSCs were identified after 1 mo recovery by GFP⁺ expression in SMA⁻Desmin⁺ cells.

FIG. 13 shows recovery from liver fibrosis is associated with a reduced number of HSCs. A. GFAP^{Cre-GFP} mice were generated by crossing GFAP^{Cre} mice with Rosa26^{flox-mTRed-Stop-flox-mGFP} mice (here labeled as Rosa26^{f/f-mTRed-mGFP} mice). Livers from GFAP^{Cre-GFP} mice (no injury n=3; CCl₄-treated n=5; recovered 1 mo n=5) were analyzed by fluorescent microscopy, and genetically labeled HSCs were visualized as membrane tagged GFP⁺ (mTRed⁻) cells. The number of GFP⁺ HSCs is expressed relative to total liver cells (100%, in white). B. Quantification of genetically labeled HSCs in liver sections. The number of genetically labeled HSCs with a history of collagen expression (Col-α2(I)^{Cre-YFP} & Col-α1(I)^{Cre-YFP} mice) or all HSCs (GFAP^{Cre-GFP} mice) is calculated as percent of total DAPI⁺ liver cells (100%).

FIG. 14 shows genetically labeled YFP⁺Desmin⁺SMA⁻ HSCs persist in livers of Collagen-α2(I)^{Cre-YFP} mice 7 weeks after withdrawal from alcohol-injury. A. A comparison of the livers of Col-α2(I)^{Cre-YFP} mice (Col-α2(I)^{Cre} mice×Rosa26^{flox-Stop-flox-YFP} mice) that were untreated (n=4), alcohol-fed (EthOH, 2 mo, n=4), or withdrawn from alcohol-feeding (7 weeks, n=8) with respect to YFP expression, Sirius Red staining, H&E and α-SMA immunohistochemistry. Representative bright field and fluorescent micrographs are shown using ×10 and ×20 objectives. B. Quantification of same four groups in (A) with respect to ratio liver weight/body weight, hydroxyproline content, Sirius Red staining, α-SMA immunofluorescence, GFP expression, collagen α1(I) mRNA level, and α-SMA mRNA level, *p<0.001, **p<0.005. C. Genetically labeled HSCs/myofibroblasts are detected in livers of mice recovered from alcohol-induced liver fibrosis. HSCs (Vitamin A⁺) from Collagen-α2(I)^{Cre-YFP} mice (no injury n=4; CCl₄-treated n=6; recovered 1 mo n=6) were analyzed by flow cytometry. Genetically labeled aHSC and iHSCs were identified by simultaneous Vitamin A⁺ and YFP⁺ expression. Dot plots are shown, p<0.01 (comparing YFP⁺ aHSC and YFP⁺ iHSCs). D. Livers from Collagen-α2(I)^{Cre-YFP} mice (no injury n=4; intragastric alcohol feeding (EthOH) n=4; recovery 7 weeks n=8) were co-stained for YFP, GFAP, Desmin, α-SMA. Genetically labeled HSCs were identified after recovery from fibrosis by YFP⁺ expression in Desmin⁺ or GFAP⁺ cells. The number of YFP⁺ HSCs is calculated relative to total HSCs (100%; alcohol-fed and recovery groups are compared, p<0.05). E. HSCs from Col-α2(1)^{Cre-YFP} mice were isolated after alcohol induced liver injury (EthOH; 2 mo.) or after 7 weeks recovery, cultured for 18 h, and analyzed for expression of Desmin, α-SMA, and synemin in genetically labeled YFP⁺ HSCs. Representative images are taken using ×40 objective.

FIG. 15 shows purified iHSCs exhibit a similar phenotype as qHSCs. HSCs from Col-α1(1)^{Cre-YFP} mice were isolated after CCl₄-injury (2 mo.) or after 1 mo recovery, cultured for 18 h, and genetically labeled YFP⁺ iHSCs were analyzed by fluorescent microscopy for expression of HSC marker Desmin, α-SMA, and neural marker synemin. Representative images are taken using ×40 objective.

FIG. 16 shows HSCs (1 mo. recovery) acquire a new phenotype distinct from aHSCs and qHSCs. A. HSCs were isolated from Collagen-α1(I)-GFP/β-actin-RFP double transgenic mice, uninjured or after recovery (7 days or 1 mo) from CCl₄, and injected intrahepatically (2.2×10⁵ cells) into 1 day old Rag2^{-/-}γc^{-/-} pups. One month later mice were gradually subjected to CCl₄ injury. Engraftment of qHSCs and HSCs 7 d and 1 mo. recovery was evaluated in each individual mice by the presence of RFP⁺GFP⁺ cells and corresponded to 50%, 78% and 80% (p<0.05), respectively. The number of activated HSCs was estimated in livers and corresponded to high (+++), intermediate (++) and low (+) and very low (±).B. HSCs were isolated from Collagen-α1(I)^{Cre-YFP} mice uninjured or after 2 weeks recovery from CCl₄-injury, and injected intrahepatically into CCl₄-treated wild type mice (n=3/group). Mice were subjected for additional 2 weeks of CCl₄, and livers were analyzed for the presence of YFP⁺Desmin⁺ HSCs by fluorescent microscopy. YFP⁺ cells were detected in all mice, due to low engraftment the results are statistically non-significant.

FIG. 17 shows genetically labeled YFP⁺ HSCs decrease collagen-α1(I)-GFP expression after 1 mo of recovery. A. Col-α1(1)^{Cre-YFP} mice were crossed with Col-GFP mice (no injury n=3; CCl₄ n=4; 1 mo recovery n=4) and livers were analyzed for YFP and GFP expression. GFP was visualized by fluorescence. YFP⁺/GFP⁺ were visualized by immunostaining with anti-GFP Ab using DAB method. The number of positive cells is calculated as percent of Desmin⁺ cells (100%, not shown). The staining is performed on the same section, images are shown using ×20 objective. B. Genetically labeled YFP⁺ HSCs decrease collagen-α1(I)-GFP expression after 1 mo of recovery. Col-α1(1)^{Cre-YFP} mice were crossed with Col-GFP mice. HSCs were isolated from livers by gradient centrifugation (no injury n=3; CCl₄ n=3, 2 w recovery n=3; 1 mo recovery n=3). Vitamin A⁺ HSCs were analyzed by flow cytometry for expression of YFP and GFP. Representative histograms are shown (representative dot plots are shown in C). The results demonstrating expression of Col-GFP and YFP in HSCs are shown as mean fluorescent intensity (mfi)±SEM, p<0.01. C. Representative dot plot are shown for HSCs isolated after CCl₄ or injury or after 2 weeks recovery from fibrosis. HSCs were isolated by gradient centrifugation, live cells (P1) were analyzed for Vitamin A expression. Vitamin A⁺ HSCs (P2) were analyzed by flow cytometry for expression of YFP and GFP (Q1-4).

FIG. 18 shows Analysis of gene expression profile of inactivated HSCs during recovery from fibrosis. A. Heat map: Genes upregulated (yellow) and downregulated (blue) are shown for distinct HSC groups. The gene expression pattern of YFP⁺ HSCs (7 days recovery) was compared to YFP⁺ aHSCs. Specific genes upregulated or downregulated in YFP⁺ HSCs (7 days recovery) versus YFP⁺ aHSCs were grouped in four clusters. B. YFP⁺ HSCs after 7 days recovery were characterized by Gene Ontology biological process annotations. YFP⁺ iHSCs (1 mo. recovery) downregulate mRNAs of Plexin Cl and Rxra, and upregulate C/EBPa. The results are relative mRNA level (average of normalized values/multiple probes/gene) obtained using Agilant microarray, *p<0.01, **p<0.001. Spp1—secreted phosphoprotein 1, Pdgfc—platelet-derived growth factor C, Bmp 5—bone morphogenic protein 5, Rxra—retinoid X receptor α, Ppara—peroxisome proliferator activated receptor α, Ece 1—endothelin converting enzyme 1, Insig 1—insulin induced gene 1. C. Expression of PPARγ, Hsp1a/b and MHCII was detected in iHSCs (1 mo recovery) using immunocytochemistry. Cell morphology (BF) and nuclei (DAPI) are also shown. Micrographs are taken with ×40 objective. D. Pathways upregulated in YFP⁺ iHSCs (1 mo.) are shown according to KEGG pathway functional enrichment analysis. iHSCs are characterized by the unique expression of 423 signature genes. Upregulated genes are marked with red stars for each pathway. The following pathways were identified for YFP⁺ iHSCs (1 mo.): Chemokine/cytokine signaling (21 genes, p<5.4e⁻⁸); Cell adhesion (11 genes, p<9.6e⁴); TLR signaling pathway (9 genes, p<7.7e⁴); Antigen presentation (7 genes, p<9.5e⁻³). E. Pathways induced in YFP⁺ HSCs (7 d) are shown according to KEGG pathway functional enrichment analysis. The following pathways were identified for YFP⁺ HSCs (7 d): ECM receptor interaction (5 genes, p<1.3e⁻²); Signaling pathways in cancer (20 genes, p<6.0e⁻³). Signaling pathways involved in ECM-receptor interaction and proliferation were strongly activated, including loss of growth inhibitory effects of TGF-β, reduced apoptosis due to p53 inhibition, activation of Wnt/β-catenin signaling pathway, and induction of pro-survival heat shock proteins.

FIG. 19 shows the role of heat shock proteins Hspa1a/b in survival of HSCs during recovery from fibrosis. A. Increased expression of Hspa1a/b mRNA in YFP⁺ HSCs after 7 days recovery was confirmed by RT-PCR, *p<0.01. B. Hspa1a/b^{-/-} HSCs lack expression of Hsp1a/b, as shown by immunostaining of HSCs isolated from Hsp1a/b−/− and wild type mice. Micrographs are taken with ×20 objective. C. Apoptosis was induced in Hsp1a/b^{-/-} and wild type HSCs by glyotoxin (25 nM) for 4 h, or by TNF-α (20 ng/ml)+Actinomycin (0.2 μg/ml) for 18 h. Cell morphology (BF), Vitamin A and apoptotic cells (TUNEL⁺ staining) are shown using ×10 objective.

FIG. 20 shows mesothelin is a new marker of activated portal fibroblasts. A. Using the Whole genome mouse microarray, the gene expression profile of activated portal fibroblasts was assessed and compared to CCl₄- and BDL-activated HSCs. Expression of mRNA of genes uniquely upregulated in aPFs is listed as “Signature genes”. Expression of PF-specific genes previously identified is shown in red. The new genes identified in our study is shown in green. The data is mRNA (fold induction), p<0.0001. B. Expression of aPF-specific new genes was confirmed by RT-PCR. and compared to other liver specific cells: Kupffer cells (KC), Endothelial cells (EC), BDL-activated PFs, BDL and CCl4-activated HSCs. Expression of mesothelin, asporin, basonuclin, calcitonin-a, uroplakin-1b mRNA was specifically induced only in activated PFs. C. Mesothelin is a marker of activated PFs. Liver tissue from non-injured, BDL- and CCl₄-injured wild type mice was stained with anti-Mesothelin Ab (Abcam). Upregulation of specific staining was detected in BDL-injured mice (versus CCl₄-injured mice). D. Human liver tissues were obtained from patients with hepatitis C, diagnosed with clinical and pathological stages of liver fibrosis (F1) and cirrhosis (F4), or no fibrosis (F0), and analyzed by immunihistochemistry for expression of human mesothelin and Sirius Red staining. Representative images are shown using ×10 objectives. Expression of mesothelin was associated with fibrotic lesions.

### DESCRIPTION OF THE INVENTION

The present disclosure relates to methods for diagnosing and treating a fibrotic condition in a subject. The subject can be any animal that exhibits fibrotic processes, preferably a mammalian subject. Mammalian subjects include, without limitation, humans, non-human primates, dogs, cats, and horses and mice.

As used herein, the term “subject” refers to an animal, typically a human (i.e., a male or female of any age group, e.g., a pediatric patient (e.g., infant, child, adolescent) or adult patient (e.g., young adult, middle-aged adult or senior adult) or other mammal, such as a primate (e.g., cynomolgus monkey, rhesus monkey); other mammals such as rodents (mice, rats), cattle, pigs, horses, sheep, goats, cats, dogs; and/or birds, that will be or has been the object of treatment, observation, and/or experiment. When the term is used in conjunction with administration of an, agent, compound or drug, then the patient has been the object of treatment, observation, and/or administration of the compound or drug.

“Treating,” “treat,” and “treatment” as used herein, refers to partially or completely inhibiting or reducing the fibrotic condition which the subject is suffering. In one embodiment, this term refers to an action that occurs while a patient is suffering from, or is diagnosed with, the fibrotic condition, which reduces the severity of the condition, or retards or slows the progression of the condition. Treatment need not result in a complete cure of the condition; partial inhibition or reduction of the fibrotic condition is encompassed by this term.

As used herein, “fibrotic condition” refers to a disease or condition involving the formation and/or deposition of fibrous tissue (or scar), e.g., excessive connective tissue builds up in a tissue and/or spreads over or replaces normal organ tissue (reviewed in, e.g., Wynn, Nature Reviews 4:583-594 (2004) and Abdel-Wahab, O. et al. (2009) Annu. Rev. Med. 60:233-45, incorporated herein by reference). In certain embodiments, the fibrotic condition involves excessive collagen mRNA production and deposition, (mostly collagen Type I). In certain embodiments, the fibrotic condition is caused, at least in part, by injury, e.g., chronic injury (e.g., an insult, a wound, a toxin, a disease). In certain embodiments, the fibrotic condition is associated with an inflammatory, an autoimmune or a connective tissue disorder. However, inflammation, damage to the blood vessels, does result in fibrosis. Activation of fibrogenic myofibroblasts is the main cause of fibrosis. For example, myofibroblasts are absent in normal tissue of non-parenchymal organs. In turn, chronic inflammation in a tissue can lead to activation of fibrogenic myofibroblasts (from different sources) in that tissue. Exemplary fibrotic tissues include, without limitation, liver tissue, lung tissue, heart tissue, kidney tissue, skin tissue, gut tissue, peritoneal tissue, bone marrow, and the like.

The term “or” is used herein to mean, and is used interchangeably with, the term “and/or”, unless context clearly indicates otherwise.

Exemplary fibrotic conditions that can be treated or prevented using the methods of the invention include, without limitation, a fibrotic condition of the lung, liver, heart, vasculature, kidney, skin, gastrointestinal tract, bone marrow, or a combination thereof.

Exemplary fibrotic conditions that can be diagnosed according to the methods of the present invention include, without limitation, any parenchymal fibroses, including acute and chronic forms of pulmonary fibrosis, interstitial lung disease, human fibrotic lung disease, liver fibrosis, cardiac fibrosis, kidney fibrosis.

In certain embodiments, the fibrosis of the liver or hepatic fibrosis is chosen from one or more of: fatty liver disease, steatohepatitis (e.g., nonalcoholic steatohepatitis (NASH), cholestatic liver disease, primary biliary cirrhosis (PBC), biliary fibrosis, cirrhosis, alcohol induced liver fibrosis, biliary duct injury, infection or viral induced liver fibrosis, congenital hepatic fibrosis, autoimmune hepatitis, or cholangiopathies (e.g., chronic cholangiopathies).

In certain embodiments, hepatic or liver fibrosis includes, but is not limited to, hepatic fibrosis associated with alcoholism, viral infection, e.g., hepatitis (e.g., hepatitis C, B or D), autoimmune hepatitis, non-alcoholic fatty liver disease (NAFLD), progressive massive fibrosis, exposure to toxins or irritants (e.g., alcohol, pharmaceutical drugs and environmental toxins such as arsenic), alpha-1 antitrypsin deficiency, hemochromatosis, Wilson's disease, galactosemia, or glycogen storage disease. In certain embodiments, the hepatic fibrosis is associated with an inflammatory disorder of the liver.

It has been previously shown that myofibroblasts, such as hepatic stellate cells (HSCs) senescence and apoptose during recovery from fibrosis. Until now, apoptosis of HSCs has not been quantified; and, therefore, it was not known that some HSCs survive and revert their phenotype. In an embodiment disclosed herein, Applicants have discovered that induction of HSCs inactivation represents a new strategy for antifibrotic therapy.

As used herein “myofibroblasts” are characterized immunophenotypically by a spindle or stellate shape, pale eosinophilic cytoplasm, expression of abundant pericellular matrix and fibrotic genes (vimentin, α-smooth muscle actin (α-SMA), non-muscle myosin, fibronectin) (33). Ultrastructurally, myofibroblasts are defined by prominent rough endoplasmic reticulum (rER), a Golgi apparatus producing collagen, peripheral myofilaments, fibronexus (no lamina) and gap junctions³³. Myofibroblasts are implicated in wound healing and fibroproliferative disorders (34-36). Studies of fibrogenesis conducted in different organs strongly suggest that resident myofibroblasts are the primary source of ECM (37). In response to fibrogenic stimuli, such as TGF-β1, myofibroblasts in all tissues express α-SMA, secrete ECM (fibronectin, collagen type I and III), obtain high contractility and change phenotype (production of the stress fibers) (38). Classical myofibroblasts differentiate from a mesenchymal lineage and, therefore, lack expression of lymphoid markers such as CD45 or CD34. Sustained injury may trigger (trans)differentiation of myofibroblasts from other cellular sources, including HSCs 1.

HSCs are perisinusoidal cells that normally reside in the Disse space and contain numerous retinoid and lipid droplets (39, 40). Under physiological conditions, HSCs exhibit a quiescent phenotype and express neural markers, such as GFAP, synamin, synaptophysin 1, and nerve growth factor receptor p75 (41, 42), secrete HGF, and store vitamin A (43). HSCs are also implicated in phagocytosis and antigen presentation (44, 45). In response to injury, quiescent HSCs lose vitamin A, acquire contractility and activate into collagen type I- and SMA-expressing myofibroblasts. Although the mechanism of HSC activation has been comprehensively studied, insights into the origin of HSCs are new (46, 47). It has been proposed that HSCs are liver resident cells and may originate from a common hepatic precursor cell (48, 49). However, similar expression of neural markers suggests that HSCs and astrocytes arise from a common progenitor during embryonic development (37, 1).

As used herein, the articles “a” and “an” refer to one or to more than one (e.g., to at least one) of the grammatical object of the article.

Disclosed herein is a diagnostic method to distinguish between different types of liver fibroses using flow cytometry to analyze and purify different subsets of fibrogenic myofibroblasts, such as resident hepatic stellate cells and portal fibroblasts (PFs) associated with a specific liver fibroses. For example, activated hepatic stellate cells were found to be the primary fibrogenic myofibroblast in CCl₄ induced liver fibrosis; whereas, activated portal fibroblasts were found to be the predominate fibrogenic myofibroblast in cholestatic liver injury. Thus, in another embodiment, Applicants have discovered that the identification of specific subsets of fibrogenic myofibroblasts in response to different kinds of fibrogenic injury allows one to study the composition of collagen producing cells for each type of fibrogenic injury and provides a definitive target for antifibrotic therapy.

As used herein “portal fibroblasts” are spindle-shaped cells that are present in most types of tissues, particularly connective tissues. These cells are of mesenchymal origin and express vimentin, but not desmin or α-SMA. Fibroblasts participate in the turnover of ECM under normal conditions (38, 50-52-21). Fibroblasts and myofibroblasts derived from portal myofibroblasts are distinct from HSCs in that they express Thy-1 (a glycophosphatidylinositol-linked glycoprotein of the outer membrane leaflet described in myofibroblasts of several organs (53, 54)), do not store retinoids, and do not express neural markers. Induced mostly by cholestatic liver injury, portal fibroblasts proliferate (though much slower than HSCs (55)) and deposit collagen (e.g. type I) around biliary tracts (56).

In still another embodiment, Applicants disclose the use of agents, compounds, or drugs, such as small molecules, nucleic acids, proteins or antibodies to target subsets of myofibroblasts associated with different types of fibroses. For example, activated HSCs may be targeted by agents or compounds that upregulate Hspa 1a/b and other signature genes described herein.

As used herein, the terms “drug,” “agent,” “compound,” and “therapeutic agent” are used interchangeably, and may include, without limitation, small molecule compounds, biologics (e.g., antibodies, proteins, protein fragments, fusion proteins, glycoproteins, etc.), nucleic acid agents (e.g., antisense, RNAi/siRNA, and microRNA molecules, etc.), vaccines, etc., which may be used for therapeutic and/or preventive treatment of a disease (e.g., liver fibrosis).

Compounds useful for treating fibrosis by inducing inactivation of a specific subset of fibrogenic myofibroblasts include PPARα agonists, such as fenofibrate, WY14643, gemfibrozil, and Ciprofibrate; PPARγ agonists, such as thizolidinediones, 15-deoxy-delta (12,14)-prostaglandin J2; compounds that induce HSP70, for example, 17-allyamino-demethoxygeldanamycin; compounds that induce Hyaluronan synthase 1 induction, for example, adiponectin; compounds that induce GATA2 activation, and compounds that induce Hspa1a/b. e.g., taurolidine, and tumor necrosis factor receptor apoptosis inducing ligand (TRAIL).

Other genes that can be targeted for antifibrotic therapy to induce inactivation of hepatic stellate cells include compounds or agents that downregulate Ssp1 and/or Pdgfc; agents or compounds that upregulate C/EBPa, BMPS, septin 4, Bambi, Hsp40, Cathepsin S and H, neural proteins: synaptogyrin 1, synaptotagmin XIII, GFAP, transcription factors: Spi-C transcription factor (spi/PU.1 related), Spi-B transcription factor (spi-1/PU.related), PU.1-IRF, IRF-1 and 3 and 5, ISRE, Stat1, Pax5, Mafk2, ISGF3-g1, BL34 regulator of G-protein signaling 1, Rnd1-Rho family GTPase 1.;

The term “upregulate” as used herein means that agent, compound or drug causes increased protein/peptide product in the target cell.

Using genetic labeling of activated HSCs (aHSCs)/myofibroblasts, Applicants demonstrate herein that some aHSCs escape cell death and revert to an inactivated phenotype (inactivated hepatic stellate cells (iHSCs)) that is similar to, but distinct from the original quiescent HSCs, including their ability to more rapidly reactivate into myofibroblasts. Thus, this newly-identified cell sub-population called is thought to be responsible for recurrent liver fibrosis. This approach for identifying iHSCs and understanding their phenotypic makeup is applicable to study fibrosis of other organs and provides an approach to identify new targets for antifibrotic therapy.

An embodiment of the invention is to induce inactivation of activated HSCs (aHSCs) to iHSCs and mitigate liver fibrosis, or prevent its recurrence.

Previously it had been thought that reversal of fibrosis is accompanied by senescence and/or apoptosis of the myofibroblasts, including aHSCs, which are responsible for the fibrosis. However, it was unknown if aHSCs myofibroblasts can escape cell death and revert to an inactive phenotype during regression of fibrosis. In an embodiment of the disclosure, Applicants disclose methods to track the cells in animals (e.g., mice and humans) involved in the diseased state.

By using a transgenic mouse system, Applicants demonstrate that different myofibroblast activation pathways are responsible for different types of liver fibrosis. For example, a majority of liver fibrosis involve mostly HSCs.

Other types of liver fibrosis, such as those that occur by blocking the common bile duct, involve both portal fibroblasts (PFs) and HSCs, however PFs play a more important role at the onset of the disease. An embodiment disclosed herein is a method to determine the most effective antifibrotic thereby by determining whether the type of liver fibrosis is caused principally by HSCs or by PFs.

Disclosed herein are specific markers that are useful for cell sorting. Myofibroblasts are aSMA⁺ Collagen Type I⁺ cells that are absent from the normal uninjured liver, rapidly emerge in fibrotic liver to produce the fibrous scar, and completely disappear with regression of liver fibrosis(1, 2). In hepatotoxic-induced liver fibrosis (such as CCl₄ or intragastric alcohol feeding), quiescent hepatic stellate cells (GFAP⁺Desmin⁺SMA⁻Col⁺ qHSCs) undergo activation to become the major source of myofibroblasts (GFAP⁺Desmin⁺aSMA⁺Col⁺ aHSCs). Disclosed herein are the use of genetic markers to address the fate of these aHSCs/myofibroblasts during regression of liver fibrosis.

Applicants show herein that survival of iHSCs requires the upregulation of pro-survival signals, such as induction of heat shock proteins(22). Two members of Hsp70 family of heat shock proteins, Hspa1a and Hspa1b(22), that play a protective role against stress-induced apoptosis(23), were strongly and transiently upregulated in HSCs after 7 days of reversal of fibrosis (when apoptosis of other aHSCs is highest) compared with the aHSCs in fibrotic liver. An embodiment disclosed herein is treating fibrosis with agents that upregulate Hspa1a/b and other heat shock proteins which are critical for transition of activated fibrogenic myofibroblasts to inactive myofibroblasts.

### Pharmaceutical Compositions, Dosage and Administration

In some embodiments, the above-described methods comprise providing agents or compounds that upregulate, for example PPARα, PPARγ, Hspa1a/b or downregulate gene products, for example, Ssp1, and Pdgfc found by Applicants to be important for inducing inactivation of fibrotic cells in vivo.

In some embodiments, the above-described methods comprise providing the agents or compounds in a pharmaceutical composition.

Pharmaceutical compositions can be formulated for administration in solid or liquid form, including those adapted for the following: oral administration, for example, drenches (e.g., aqueous or non-aqueous solutions or suspensions), tablets (e.g., those targeted for buccal, sublingual, and systemic absorption), capsules, boluses, powders, granules, pastes for application to the tongue; parenteral administration, for example, by subcutaneous, intramuscular, intravenous or epidural injection such as, for example, a sterile solution or suspension, or sustained-release formulation; topical application, for example, as a cream, ointment, or a controlled-release patch or spray applied to the skin; intravaginally or intrarectally, for example, as a pessary, cream or foam; sublingually; ocularly; transdermally; pulmonarily; or nasally.

Pharmaceutically acceptable excipients include any and all fillers, binders, surfactants, disintegrants, sugars, polymers, antioxidants, solubilizing or suspending agents, chelating agents, preservatives, buffering agents and/or lubricating agents, or combinations thereof, as suited to the particular dosage form desired and according to the judgment of the formulator. Remington's Pharmaceutical Sciences, Sixteenth Edition, E. W. Martin (Mack Publishing Co., Easton, Pa., 1980) discloses various pharmaceutically acceptable excipients used in preparing compositions and known techniques for the preparation thereof. Except insofar as any conventional carrier medium is incompatible with the compounds disclosed herein, such as by producing any undesirable biological effect or otherwise interacting in a deleterious manner with any component of the composition, its use is contemplated to be within the scope of this invention. In general, the compositions are prepared by uniformly and intimately bringing into association the compounds or agents described above with one or more excipients and then, if necessary, shaping the product.

When the agent or compound is administered to humans or animals it can be given per se or as a pharmaceutical composition containing, for example, about 0.1 to 99%, or about 10 to 50%, or about 10 to 40%, or about 10 to 30%, or about 10 to 20%, or about 10 to 15% of the agent or compound in combination with a pharmaceutically acceptable excipient.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

Actual dosage levels of the agent or compound in the pharmaceutical compositions can be varied so as to obtain an amount of the active ingredient which is effective to achieve the desired therapeutic response for a particular subject, composition, and mode of administration, without being toxic to the subject.

The selected dosage level will depend upon a variety of factors including, for example, the activity of the particular agent or compound employed, the route of administration, the time of administration, the rate of excretion or metabolism, the rate and extent of absorption, the duration of the treatment, other drugs, compounds or materials used in combination with the agent or compound, the age, sex, weight, condition, general health and prior medical history of the subject, and other similar factors well known in the medical arts.

In general, a suitable daily dose of a compound or agent will be that amount which is the lowest dose effective to produce a therapeutic effect. Such an effective dose will generally depend upon the factors described above. Generally, oral, intravenous and subcutaneous doses of the agent or compound for a subject, when used for the indicated effects, will range from about 0.0001 mg to about 100 mg per day, or about 0.001 mg to about 100 mg per day, or about 0.01 mg to about 100 mg per day, or about 0.1 mg to about 100 mg per day, or about 0.0001 mg to about 500 mg per day, or about 0.001 mg to about 500 mg per day, or about 0.01 mg to about 500 mg per day, or about 0.1 mg to about 500 mg per day.

“Therapeutically effective amount,” or “therapeutic effect,” as used herein, refers to a minimal amount or concentration of an agent, compound and/or drug that, when administered alone or in combination, is sufficient to provide a therapeutic benefit in the treatment of the condition, or to delay or minimize one or more symptoms associated with the condition. The term “therapeutically effective amount” can encompass an amount that improves overall therapy, reduces or avoids symptoms or causes of the condition, or enhances the therapeutic efficacy of another therapeutic agent. The therapeutic amount need not result in a complete cure of the condition; partial inhibition or reduction of the fibrotic condition is encompassed by this term.

In some embodiments, the agent or compound prevents the condition or can be used at prophylactically effective amount.

As used herein, unless otherwise specified, the terms “prevent,” “preventing” and “prevention” refers to an action that occurs before the subject begins to suffer from the condition, or relapse of such condition. The prevention need not result in a complete prevention of the condition. Partial prevention or reduction of the fibrotic condition is encompassed by this term.

As used herein, unless otherwise specified, a “prophylactically effective amount” of an agent, compound and/or drug, when administered alone or in combination, prevent the condition, or one or more symptoms associated with the condition, or prevent its recurrence. The term “prophylactically effective amount” can encompass an amount that improves overall prophylaxis or enhances the prophylactic efficacy of another prophylactic agent. The prophylactic amount need not result in a complete prevention of the condition; partial prevention or reduction of the fibrotic condition is encompassed by this term.

The subject receiving the treatment can be any animal in need, including primates (e.g. humans), equines, cattle, swine, sheep, poultry, dogs, cats, mice and rats.

The agent or compound can be administered daily, every other day, three times a week, twice a week, weekly, or bi-weekly. The dosing schedule can include a “drug holiday,” i.e., the drug can be administered for two weeks on, one week off, or three weeks on, one week off, or four weeks on, one week off, etc., or continuously, without a drug holiday. The agent or compound can be administered orally, intravenously, intraperitoneally, topically, transdermally, intramuscularly, subcutaneously, intranasally, sublingually, or by any other route.

### Combination Therapies

The agents or compounds described above can be administered in combination with one or more therapeutic agents. Exemplary therapeutic agents include, but are not limited to, antifibrotics, corticosteroids, anti-inflammatories, immunosuppressants, chemotherapeutic agents, anti-metabolites, and immunomodulators.

By “in combination with,” it is not intended to imply that the therapeutic agent and agent or compound must be administered at the same time and/or formulated for delivery together, although these methods of delivery are within the scope of the invention. The agent or compound can be administered concurrently with, prior to, or subsequent to, one or more other additional agents. In general, each therapeutic agent will be administered at a dose and/or on a time schedule determined for that particular agent. In will further be appreciated that the therapeutic agent utilized in this combination can be administered together in a single composition or administered separately in different compositions. The particular combination to employ in a regimen will take into account compatibility of the antifibrotic agent or compound with the agent and/or the desired therapeutic effect to be achieved.

In general, it is expected that additional therapeutic agents employed in combination be utilized at levels that do not exceed the levels at which they are utilized individually. In some embodiments, the levels utilized in combination will be lower than those utilized individually. The determination of the mode of administration and the correct dosage for each agent or combination therapy is well within the knowledge of the skilled clinician.

In embodiments where two agents are administered, the two agents can be administered concurrently (i.e., essentially at the same time, or within the same treatment) or sequentially (i.e., one immediately following the other, or alternatively, with a gap in between administration of the two). In some embodiments, the antifibrotic agent or compound is administered sequentially (i.e., after the first therapeutic).

Suitable therapeutics for use in combination with the compounds for inducing inactivation of fibrogenic myofibroblasts for treatment of liver fibrosis includes, without limitation, adefovir dipivoxil, candesartan, colchicine, combined ATG, mycophenolate mofetil, and tacrolimus, combined cyclosporine microemulsion and tacrolimus, elastometry, everolimus, FG-3019, Fuzheng Huayu, GI262570, glycyrrhizin (monoammonium glycyrrhizinate, glycine, L-cysteine monohydrochloride, interferon gamma-1b, irbesartan, losartan, oltipraz, ORAL IMPACT®., peginterferon alfa-2a, combined peginterferon alfa-2a and ribavirin, peginterferon alfa-2b (SCH 54031), combined peginterferon alpha-2b and ribavirin, praziquantel, prazosin, raltegravir, ribavirin (REBETOL®., SCH 18908), ritonavir-boosted protease inhibitor, pentoxyphilline, tacrolimus, tauroursodeoxycholic acid, tocopherol, ursodiol, warfarin, and combinations thereof.

The invention now being generally described, it will be more readily understood by reference to the following examples, which are included merely for purposes of illustration of certain aspects and embodiments of the present invention, and are not intended to limit the invention.

### EXAMPLES

### Methods

Mice:

Expression of collagen Type I in real time was studied using reporter Col-GFP mice(25). Cell fate mapping of aHSCs was studied using collagen-α2(I)^{Cre} (26) and collagen-α1(I)^{Cre} and tamoxifen-inducible collagen-α2(I)^{ER-Cre} crossed to Rosa26^{flox-Stop-flox-YFP} mice (or Rosa26^{flox-mTRed-Stop-flox-mGFP} mice, Jackson Labs). GFAP^{Cre} mice are used to determine the total number of HSCs.

Liver Fibrosis:

Liver fibrosis was induced in mice by intragastric gavage with carbon tetrachloride, CCl₄ (at 16×1:4 dilution in 100 μl of corn oil) over 2 months(8), or intragastric ethanol feeding combined with Western diet (for 2 months)(27). Reversal of liver fibrosis was studied 1 month after CCl₄ cessation, and 7 weeks after withdrawal from alcohol feeding. Recurrent injury in Col-GFP mice was induced for 1 mo with CCl₄ (8×1:4). Liver injury in Rag^{-/-}γc^{-/-} and Hspa1a/b^{-/-} mice was gradually induced with CCl₄ (4×1:16; 2×1:8; 2×1:4) for 1 month. Collagen content is estimated by Hydroxyproline, Sirius Red staining. For PF studies Liver injury was induced in mice by intragastric gavage with carbon tetrachloride CCl₄ (1:4 dilution in corn oil, 60 μl×14 injections) or ligation of the common bile duct (3 weeks)

Adoptive Transfer of HSCs into Rag^{-/-}γc^{-/-} Mice.

Primary HSCs were isolated from Collagen-α1(I)-GFP/β-actin-RFP double transgenic mice, uninjured or after cessation of CCl₄-induced injury (7 days or 1 mo) and adoptively transferred (2.2×10⁵ cells) into 1 day old Rag2^{-/-}γc^{-/-} pups by intrahepatic injection. One month later mice were gradually subjected to CCl₄-induced liver injury.

Isolation of Non-Parenchymal Cell Fraction and Primary HSCs:

Livers are perfused and digested using pronase/collagenase and gradient centrifugation method, as previously described (8). Freshly isolated HSCs were analyzed by flow cytometry, or cultured in DMEM (Gibco-BRL)+10% FCS, 2 mM L-glutamine+antibiotics. For PF studies Livers were perfused and digested using pronase/collagenase method. Singe-cell suspensions were centrifuged at 50 g for 5 minute to pellet the hepatocyte fraction. The remaining nonparenchymal cell fraction supernatant was collected and contained hepatic myofibroblasts (HSCs, portal fibroblasts and others), Kupffer cells, BM inflammatory cells and hepatic endothelial cells (HEC).

Flow Cytometry:

Flow cytometry was based on simultaneous detection of collagen-α1(I)-GFP and Vitamin A (autofluorescent signal detected by UV laser in Col-GFP mice. Phenotyping of the non-parenchymal fraction isolated from liver injured Col-GFP mice was performed on Canto (BD Bioscience Flow Cytometry Systems, BD). Activated myofibroblasts were visualized by GFP expression (488 nm) using argon laser, and Vitamin A⁺ cells were visualized by autofluorescent signal (405 nm) detected by violet laser. Thy1.1-PE antibody (eBioscience) was used to distinguish PFs from HSCs. Cell sorting was performed on a MoFlo (Beckman Colter). Activated myofibroblasts were visualized by GFP expression (488 nm) using LYT-2005 laser (iCYP Visionary Bioscience Inc), and Vitamin A⁺ cells were visualized by autofluorescent signal (350 nm) detected by UV laser (JDSU-Excyte).

Immunofluorescence and Immunohistochemistry.

Formalin-fixed frozen livers or isolated cells (fixed in 5% Paraformaldehyde in PBS) were stained with anti-desmin Ab (Thermo Scientific), anti-GFAP (Dako), anti-GFP Ab (Abcam), anti-SMA Ab (Abcam), anti-MHC II Ab, PECAM-1 (eBioscience), anti-PPARγ (Santa Cruz), anti-Hspa1a/b Ab (gift of Dr. Dillman) or isotype controls. Nuclei are stained with DAPI. Immunohstochemistry is performed using DAB staining (Vector). For PF studies Immunohstochemistry was performed using DAB staining (Vector), and counterstaining with Hematoxilin, Isolated cells were fixed in 5% Paraformaldehyde in PBS and stained with anti-Mesothelin antibody using MOM kit (Vector).

Generation of Transgenic Col-α1(I)^{Cre} mice. Collagen-α1(I)^{Cre} (Col-α1(I)^{Cre}) transgenic mice express Cre under the control of collagen-α1(I) promoter/enhancer. The transgenic construct was generated using pGL3(R2.1) basic Vector (Promega, Madison). Collagen-α1 (I) promoter/enhancer was inserted into the plasmid using Kpn I and Bgl II restriction enzymes. Cre was inserted into EcoRI-EcoRI site. The transgenic construct was excised with Kpn I and Sal I unique restrictions enzymes and microinjected into fertilized C57BL/6J×CBA F1 hybrid embryos, which were implanted in pseudo-pregnant Swiss Webster foster mothers. The offspring (founders) were genotyped by PCR of genomic DNA for primers detecting Cre. All animal experiments were approved by the UCSD Institutional Animal Care and Use Committee.

Intragastric Ethanol Feeding Model of Liver Fibrosis, and Withdrawal from Ethanol Feeding.

Col-α1(I)^{Cre-YEP} male and female (13 wks old) mice were first fed ad libitum “Western diet”, a solid diet high in cholesterol and saturated fat (HCFD: 1% w/w cholesterol, 21% Cal lard, 17% Cal corn oil) for 2 weeks. The mice were then operated for implantation of gastric catheters for intragastric feeding of liquid high fat diet (36% Cal corn oil) plus ethanol or isocaloric dextrose at 60% of daily caloric intake for 8 weeks for males and 10 weeks for females(27-29). During this intragastric feeding period, the mice continued to consume ad libitum HCFD for missing 40% of calories. Ethanol dose was increased from 19 to 32 g/kg/day. Similar level of fibrosis was achieved in males and females. Withdrawal from alcohol began by replacing HCFD with regular chow and gradually decreasing the ethanol dose within 7 days. The catheter was then cut off just above the dorsal exit site, and the animals were allowed to recover from alcoholic liver fibrosis for 7 weeks prior to sacrificing and isolation of hepatic stellate cells.

Adoptive Transfer of Primary HSCs into the Wild Type Mice.

Primary HSCs were isolated from Collagen-α1(I)^{Cre-YFP} mice, uninjured or 2 weeks after cessation of CCl₄-induced injury and adoptively transferred (intrahepatically) into the wild type C57Bl6 male mice (12 w old, males, n=3/group), pre-treated with CCl₄ (4×1:4 dilution). HSCs from a single donor were transferred into one recipient mouse. Following the transfer, mice continued to receive CCl₄ (4×1:4 dilution) for 2 weeks to induce liver injury (see. FIG. 6B).

Whole Mouse Genome Gene Expression Microarray:

The gene expression profile of HSCs was studied using Whole Mouse Genome Microarray (Agilent). For this purpose, Vitamin A⁺YFP⁺ and Vitamin A⁺YFP⁻ HSCs were sort purified from Collagen-α2(I)^{Cre-YFP} mice (8 w old) with no injury, after CCl₄ (2 mo.), and after 7 days or 1 mo recovery from CCl₄. In addition, Vitamin A⁺GFP⁺ qHSCs were sort purified from Collagen-α1(I)-GFP mice at day 14 postnataly. mRNA was purified using RNAeasy columns (Qiagen, Valencia, Calif.), 160 ng of purified RNA per sample was labeled using the LRILAK PLUS, two color low RNA input Linear Amplification kit and hybridized to a Whole Mouse Genome Microarray 4×44K 60 mer slide according to the manufacturer's instructions (Agilent, Santa Clara, Calif.). Slides were scanned using the Agilent GZ505B Scanner and analyzed using the Gene Spring Software (Agilent). Hierarchical clustering of gene expression values was performed using Cluster 3.0 (http://bonsai.hgc.jp/˜mdehoon/software/cluster/software.htm, (30)) using the correlation coefficient as the similarity metric, and average linkage when merging nodes during tree building. Clustering was performed on genes expressed in at least one condition (>9 log₂ intensity value) to remove absent genes and genes exhibiting a standard deviation greater than 0.75 among log₂ intensity values to remove genes with constant expression. Hierarchical clustering results were visualized using Java Tree View (http://jtreeview.sourceforge.net/, (31)). Differentially regulated genes were defined as those with significant absolute expression (>9 log₂ intensity value) and exhibiting 2-fold compared to the maximal value in all other samples. Gene ontology and KEGG pathway functional enrichment analysis was performed using DAVID (http://david.abcc.ncifcrf.gov, (32)).

Quantitative RT-PCR:

Total RNA was isolated from purified HSCs using RNeasy columns (Qiagen, Valencia, Calif.). First strand cDNA was synthesized using SuperScript III and random hexamers (Invitrogen, Carlsbad, Calif.). Samples were run in 20 ul reactions using an AB1 7300 (Applied Biosystems, Foster City, Calif.). SYBR Green oligonucleotides were used for detection and quantification of genes. Gene expression levels were calculated after normalization to the standard housekeeping gene 18S using the ΔΔ CT method as described by the manufacturer (Invitrogen, Carlsbad, Calif.), and expressed as relative mRNA levels compared with control. The results are represented as average±SEM, p<0.0001

Apoptosis of aHSCs.

Apoptosis was induced in serum starved Hsp1a/b−/− and wild type HSCs by glyotoxin (25 nM for 4 h) or TNF-α (20 ng/ml for 18 h)+Actinomycin (0.2 μg/ml) (13, 14). Cell apoptosis was accessed by TUNEL⁺ staining (Roche) and immunostaining for cleaved caspase-3 (Cell technologies. Inc.).

### Example 1

### Regression of Liver Fibrosis is Accompanied by Loss of Myofibroblasts

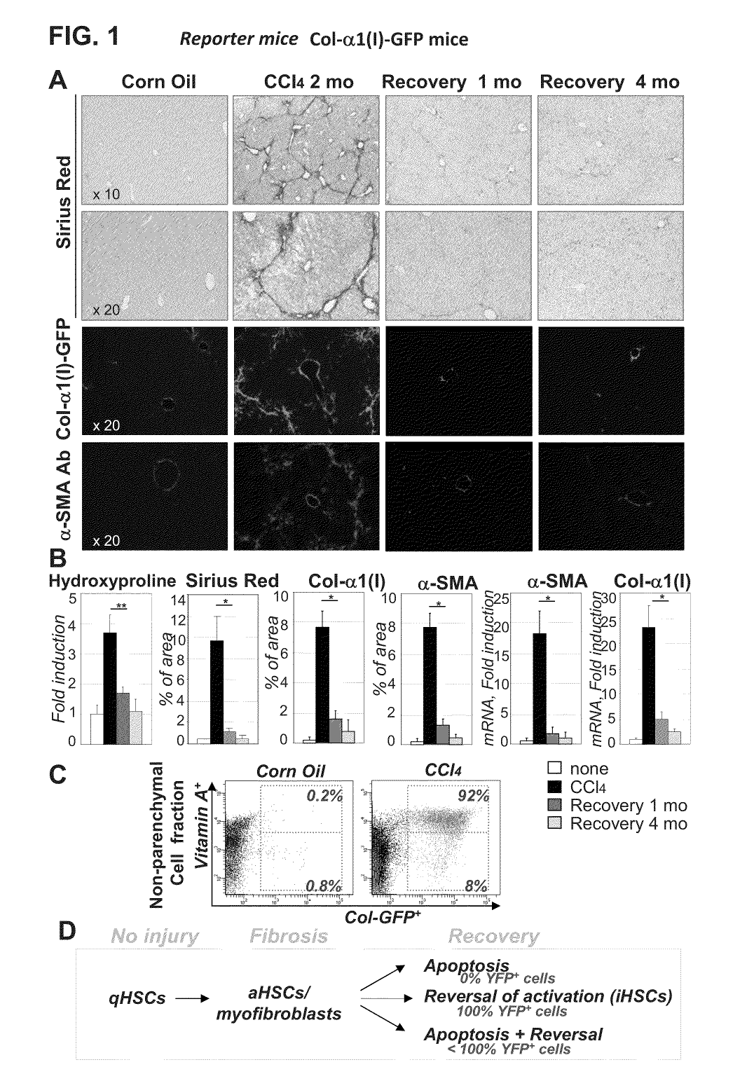

A study was designed to determine the fate of aHSCs/myofibroblasts (α-SMA⁺ColI⁺ cells) during regression of hepatic fibrosis. For this purpose, reporter Col-GFP mice, expressing collagen-α1(I) promoter/enhancer-driven GFP, were subjected to CCl₄-induced liver injury for 2 months. After cessation of the toxic agent, mice recuperated for 1 or 4 months, and regression of liver fibrosis was evaluated by measuring collagen deposition and myofibroblast number (FIG. 1A-B). CCl₄-treated mice developed severe fibrosis with activated myofibroblasts (FIG. 1A-B), that decreased markedly after 1 mo. and 4 mo. of recovery. After 1 mo recovery, hydroxyproline levels and expression of fibrogenic genes collagen-α1(I) and α-SMA were significantly decreased, compared with CCl₄ treated mice (7.8±1.2% Col-GFP and 8±1.5% α-SMA, FIG. 1B), confirming that CCl₄-activated myofibroblasts disappear during recovery from liver fibrosis. Thus, Col-GFP mice undergo regression of liver fibrosis so that 1 month of recovery is appropriate to study the fate of aHSCs/myofibroblasts.

### Example 2

### Hepatic Stellate Cells are the Major Source of CCl₄-Activated Myofibroblasts

The contribution of aHSCs to liver myofibroblasts in CCl₄-treated Col-GFP mice was determined using flow cytometry of the isolated non-parenchymal liver cell fraction, which contains aHSCs/myofibroblasts, inflammatory cells, and endothelial cells(8). Myofibroblasts were identified by Col-GFP expression, and HSCs were identified by Vitamin A expression(1, 4, 8) (detected at 405 nm as an autofluorescent signal quenched by a violet laser, FIG. 1C and FIG. 6). 92±3% of GFP cells co-expressed Vitamin A, demonstrating that aHSCs represent the major population of fibrogenic myofibroblasts in CCl₄-injured liver, as predicted by previous qualitative studies(9). Therefore, aHSCs can be genetically labeled based on specific upregulation of type I collagen expression (FIG. 7) in CCl₄-induced liver fibrosis, since other cellular sources do not make a significant contribution to the myofibroblast population.

Collagen-GFP mice were also subjected to cholestatic (BDL) liver injury. Using this model activation of portal fibroblasts (PFs) prevailed over HSC in response to BDL. Moreover, BDL-induced PFs correlated with their increased activation (versus HSCs) and expression of fibrogenic genes (α-SMA, collagen-α1(I), TIMP-1, TGF-β1). Fibrogenic properties exhibited by BDL-induced PFs were comparable to that in CCl₄-induced HSCs.

### Example 3

### Some aHSCs Apoptose During Regression of Liver Fibrosis

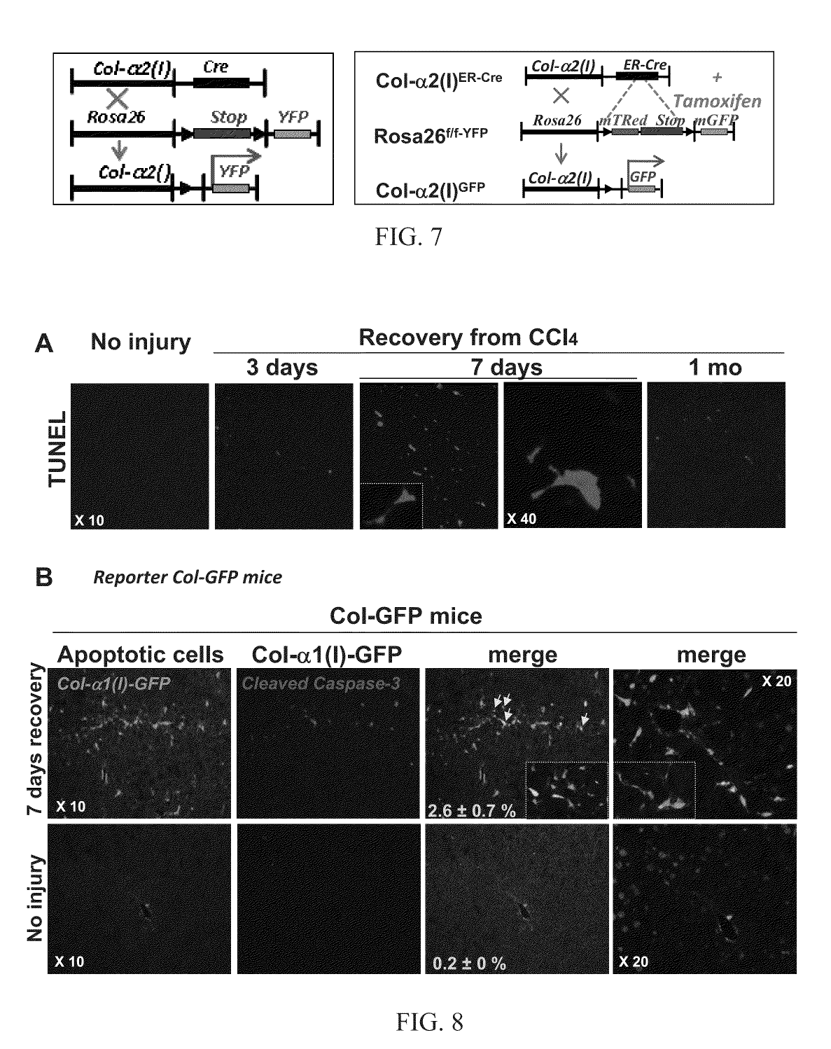

The disappearance of aHSCs/myofibroblasts during regression of liver fibrosis may result from either cell death by senescence(3) and apoptosis(2), inactivation (iHSCs), or both (FIG. 1D). Apoptosis of HSCs during regression of liver fibrosis is well documented(2). In agreement, we detected apoptotic aHSCs/myofibroblasts (2.6±0.7%) by co-localization of cleavable caspase-3⁺ and GFP⁺ cells in the livers of Col-GFP mice 7 days after CCl₄ cessation, when apoptosis of hepatic cells was highest (FIG. 8). Overall, early (7 days) recovery from liver fibrosis is accompanied by apoptosis of some aHSCs/myofibroblasts.

### Example 4

### Genetically Labeled aHSCs/Myofibroblasts Persist in the Liver after 1 mo of Recovery from CCl₄

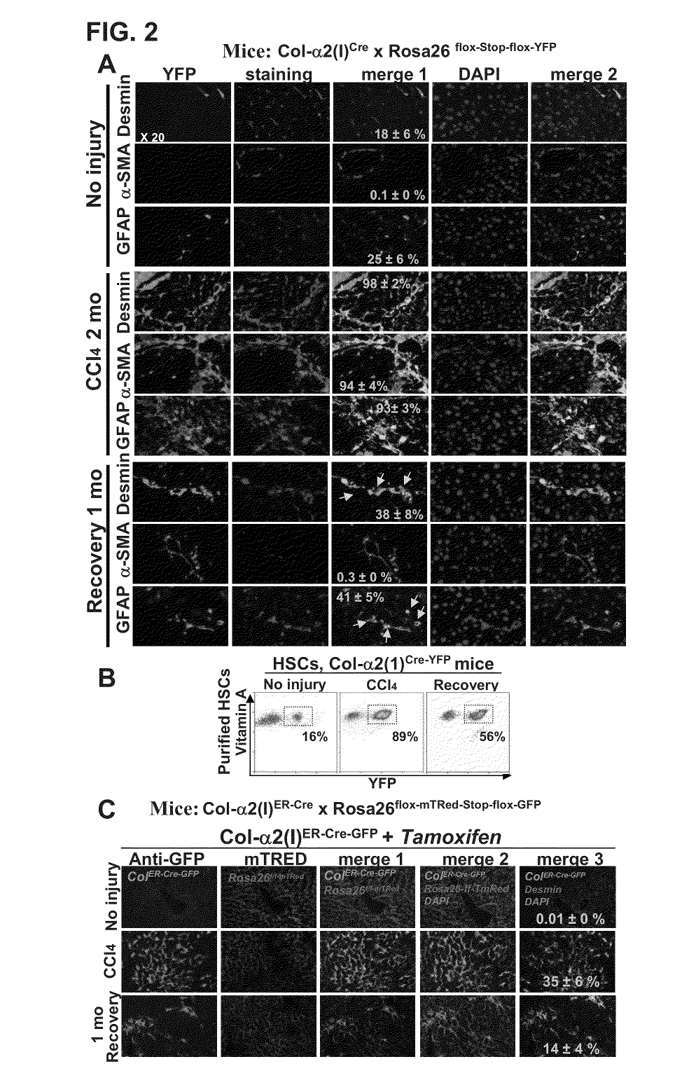

To determine if some liver myofibroblasts survive the regression of fibrosis, Col-α2(I)^{Cre-YFP} mice (Collagen-α2(I)^{Cre}×Rosa26^{flox-Stop-flox-YFP} mice, see FIG. 7) were treated with CCl₄ (2 mo), allowed to recover (1 mo) and then were analyzed for the persistence of genetically labeled YFP⁺ cells (FIG. 2A). HSCs were identified by expression of GFAP and Desmin, and aHSCs/myofibroblasts were detected by expression of α-SMA. 98±2% of HSCs were activated (expressed YFP) in response to CCl₄ treatment, and YFP expression was detected in 94±4% of myofibroblasts (α-SMA⁺). Although myofibroblasts had completely disappeared in livers after 1 mo recovery, YFP⁺ cells surprisingly persisted. In particular, expression of YFP was detected in 38±8% of Desmin⁺ and 41±5% of GFAP⁺ cells, consistent with being HSCs that had been previously activated (FIG. 2A).

The immunoshistochemistry (FIG. 2A) and flow cytometry (FIG. 2B) of gradient purified HSCs from Col-α2(I)^{YFP} mice identified three HSC phenotypes 1) quiescent (qHSCs, Vitamin A⁺ YFP⁻ α-SMA⁻), 2) activated (aHSCs, Vitamin A⁺ YFP⁺α-SMA⁺), and 3) inactivated (iHSCs, Vitamin A⁺ YFP⁺ α-SMA⁻). After recovery from fibrosis, 56±4% of HSCs co-expressed YFP⁺ and Vitamin A⁺, indicating that these iHSCs had a history of Type I expression but reverted to an inactivated phenotype (FIG. 2B).

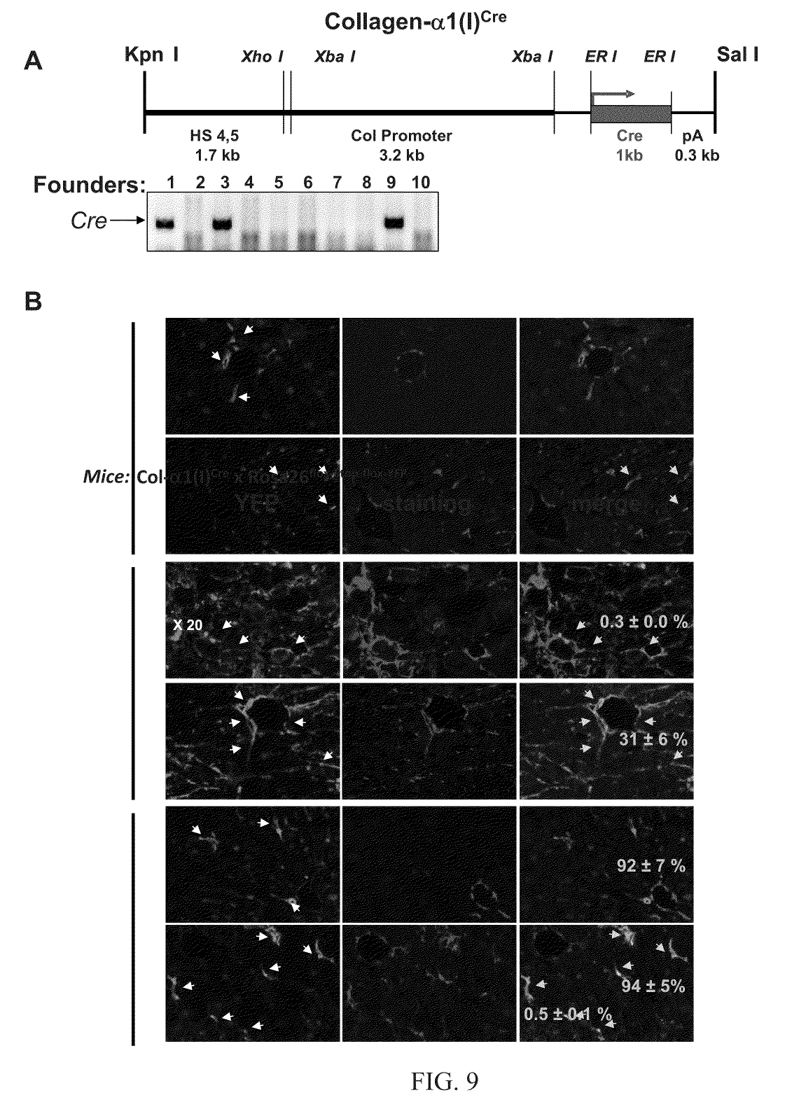

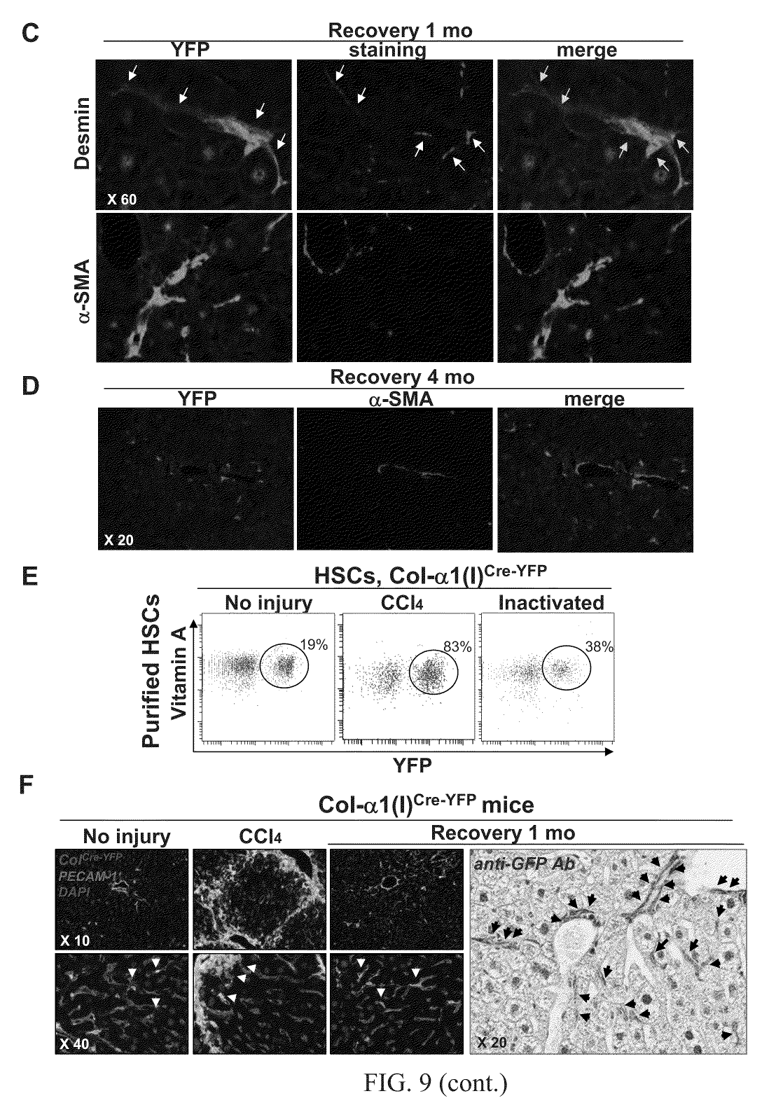

Collagen-α2(I) and -α1(I) form a triple helix to produce collagen Type I and are co-expressed in aHSCs/myofibroblasts (10). To provide independent confirmation of the above findings, we used Col-α1(1)^{Cre-YFP} mice, generated by crossing collagen-α1(I)^{Cre} mice (FIG. 9A) with Rosa26^{flox-Stop-flox-YFP} mice. As expected, CCl₄ treatment of Col-α1(1)^{Cre-YFP} mice produced aHSCs (Desmin⁺YFP⁺α-SMA⁺ cells; FIG. 9B-C). While α-SMA⁺ myofibroblasts were no longer detected in livers after 1 mo recovery, 37±9% of Desmin⁺ HSCs still expressed YFP. In fact, genetically labeled YFP⁺ HSCs persisted after 4 mo recovery (FIG. 9D). Similarly, flow cytometry demonstrated that 38±7% of YFP⁺Vitamin A⁺ HSCs expressed YFP after 1 mo recovery, compared to 83±6% of YFP⁺ VitaminA⁺ aHSCs in fibrotic liver (FIG. 9E). In the recovered liver, these iHSCs resided in the peri-sinusoidal space of Disse and exhibited a stellate shape (FIG. 9F).

### Example 5

### HSCs Transiently Express Collagen Type I During Development

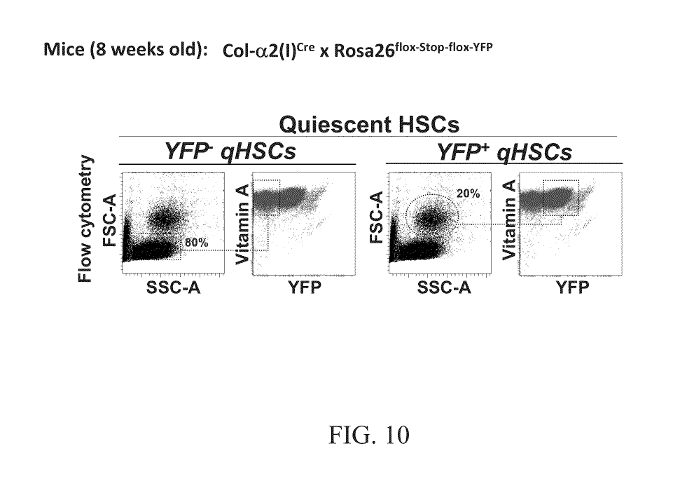

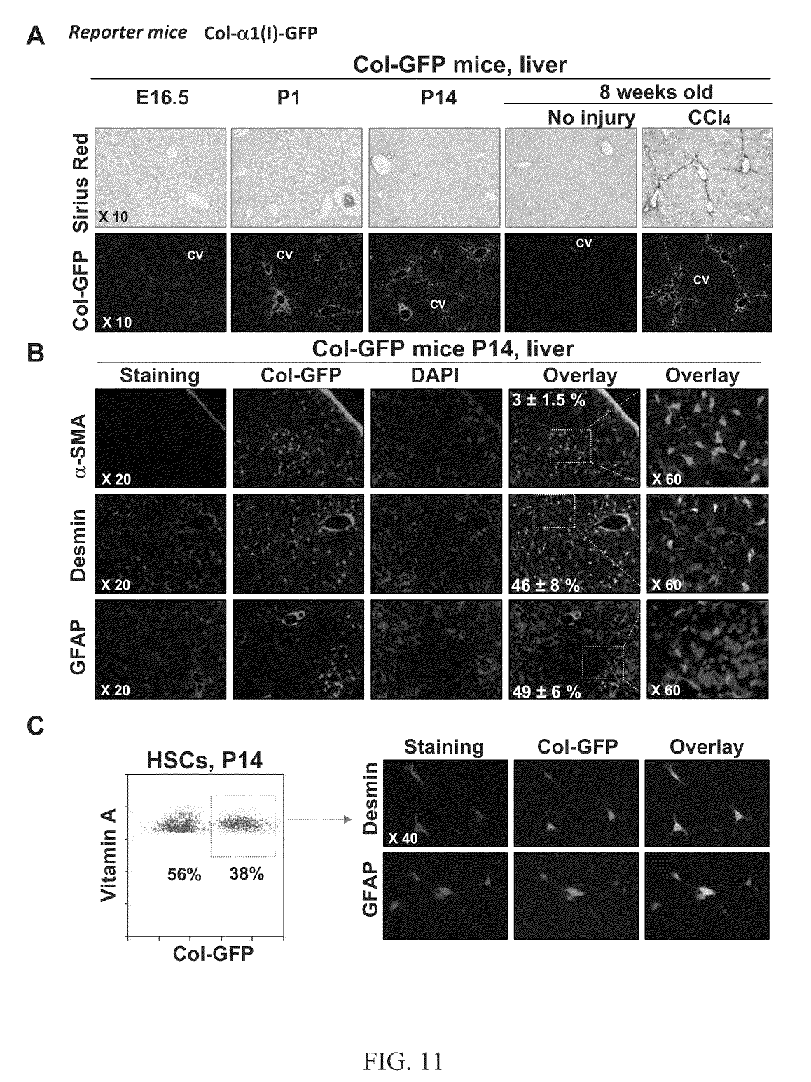

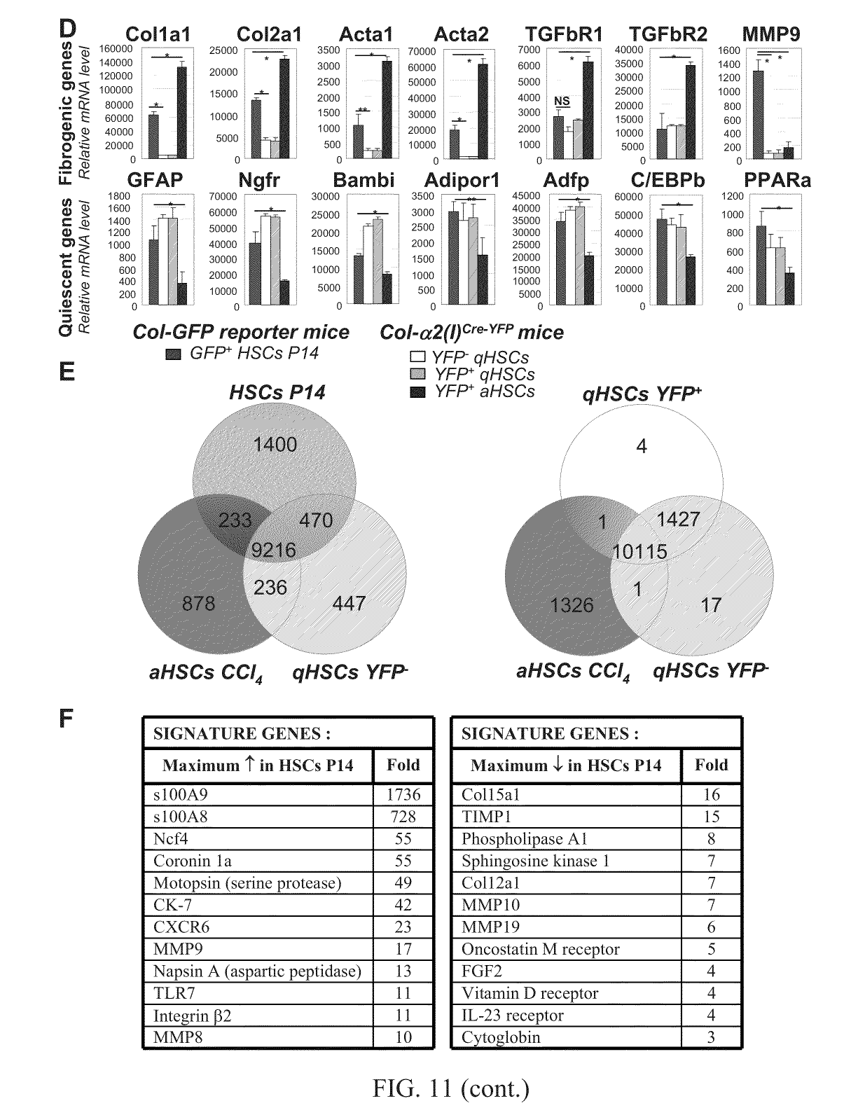

Detection of YFP⁺ qHSCs in Col-α2(1)^{Cre-YFP} and Col-α1(1)^{Cre-YFP} in adult livers prior to injury (FIG. 2B, FIGS. 9E & 10) may reflect transient collagen gene expression activating Cre during development. To prove this hypothesis, expression of collagen-α1(I) in real time was examined in livers of Col-GFP mice during embryogenesis (FIG. 10). Indeed, transient expression of collagen-α1(I)-GFP was detectable in HSCs, identified by Vitamin A, Desmin and GFAP expression, between embryonic E16.5—postnatal day 14 (P14, FIG. 11A). At postnatal day 14, 46±8% of HSCs upregulated collagen-α1(I)-GFP in real time but lacked α-SMA expression (FIG. 11B). These GFP⁺ HSCs did not exhibit characteristics of myofibroblasts (FIG. 11C-D), but were more similar to qHSCs than to aHSCs (FIG. 11E-F).

The fate of embryonic collagen⁺ HSCs was examined in adult Col-α2(1)^{Cre-YFP} mice (8 w old). Consistent with our findings, YFP⁺ qHSCs with a history of collagen expression and YFP⁻ qHSCs had identical gene expression profiles characteristic of a quiescent phenotype (FIG. 11E).

### Example 6

### Tamoxifen-Induced Genetic Labeling of aHSCs/Myofibroblasts in Adult Mice Confirmed Their Persistence in the Liver after 1 mo of Recovery from CCl₄

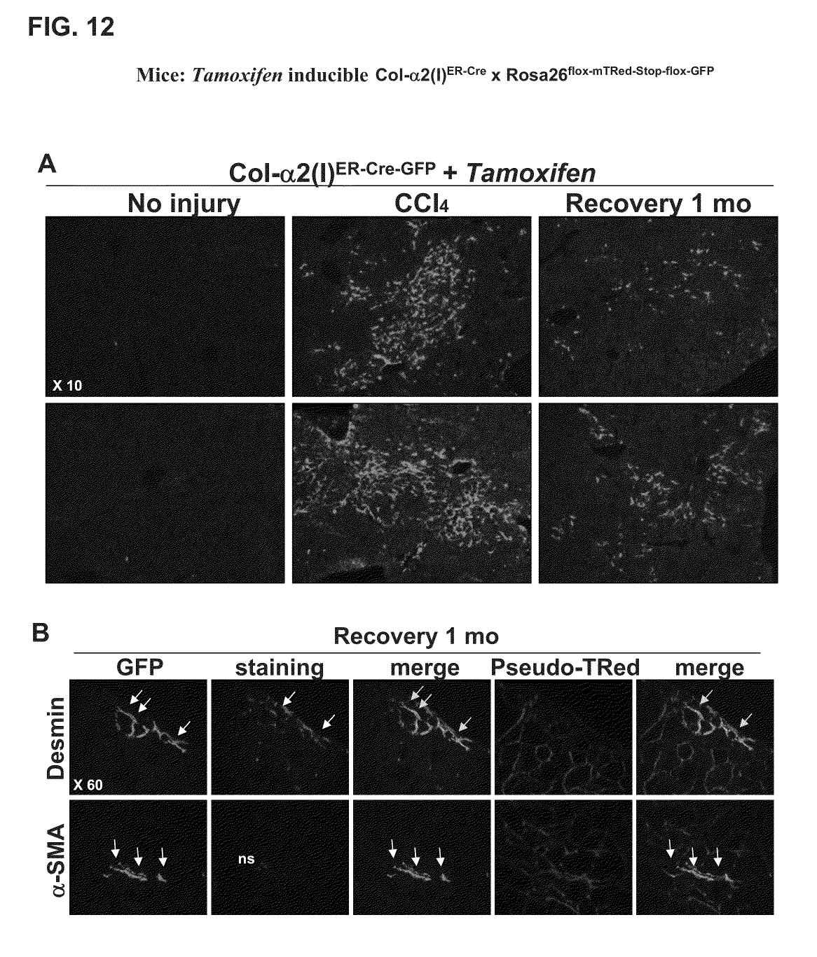

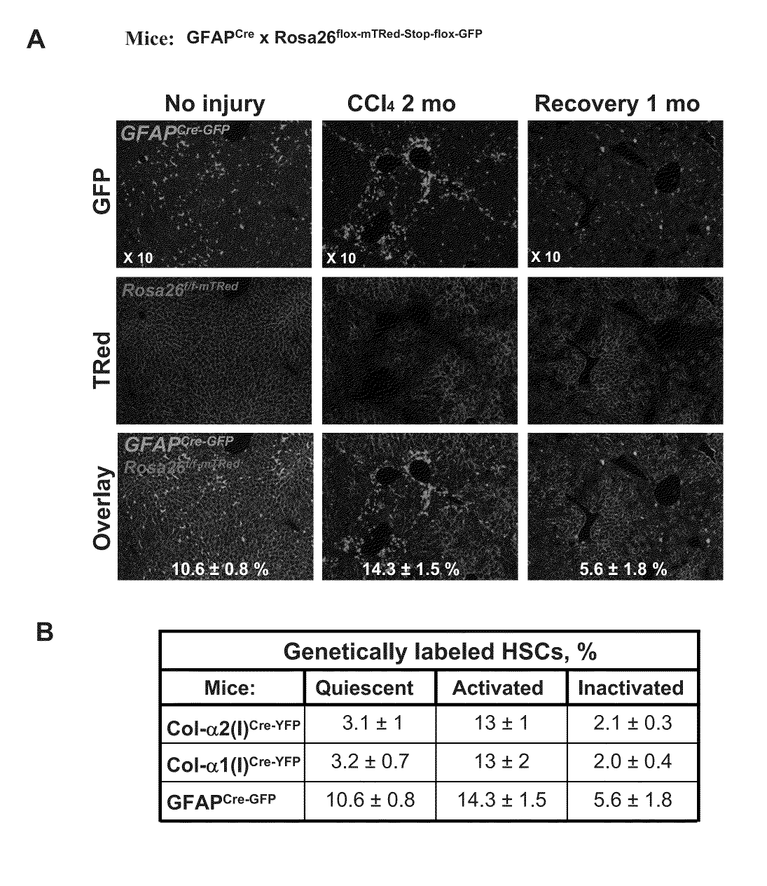

Tamoxifen-inducible Col-α2(1)^{ER-Cre-GFP} mice were generated by crossing Col-α2(1)^{ER-Cre} mice×Rosa26^{flox-mTRed-Stop-flox-mGFP} mice (FIG. 7). Genetic labeling of HSCs was achieved in adult CCl₄-treated Col-α2(1)^{Er-Cre-GFP} mice by daily tamoxifen administration during the last week of CCl₄ treatment (FIG. 12A). Genetically labeled aHSCs were visualized by loss of mTRed expression and gain of GFP expression upon Cre-loxP recombination. 35±6% of Desmin⁺ HSCs expressed GFP after CCl₄, and 14±4% of HSCs were still GFP⁺ after 1 mo. recovery (FIG. 2C), confirming that CCl₄-activated HSCs (and their progeny) persist in the liver after regression of fibrosis. Consistently, GFP⁺ iHSCs expressed Desmin, but not α-SMA (FIG. 12B). Thus, three independent transgenic mice demonstrated that aHSCs/myofibroblasts revert to an inactive phenotype during regression of fibrosis.

### Example 7

### Livers Recovering from Fibrosis have Fewer HSCs

To quantify the number of HSCs during fibrosis and its regression, we generated GFAP^{Cre-GFP} mice (GFAP^{Cre} mice×Rosa26^{flox-Stop-mTRed-flox-mGFP} mice, FIG. 13A). In uninjured mice, qHSCs were distributed throughout the hepatic acinus and represented 10.6±0.8% of total liver cells. CCl₄ induced HSC activation, proliferation (14.3±1.5% of total liver cells), and accumulation of aHSCs in the pericentral area. One month after recovery, the number of HSCs was reduced (5.6±1.8% of total liver cells), and the distribution of HSCs was again similar to qHSCs. Based on immunostaining for GFAP after recovery from fibrosis in Col-α2(I)^{Cre-YFP} and Col-α1(I)^{Cre-YFP} mice (FIG. 2B, FIG. 9E), iHSCs constitute 2% of total liver cells in the recovered liver (FIG. 13B).

### Example 8

### Genetically Labeled aHSCs/Myofibroblasts Persist in the Liver after 7 Weeks of Recovery from Alcohol-Induced Liver Fibrosis

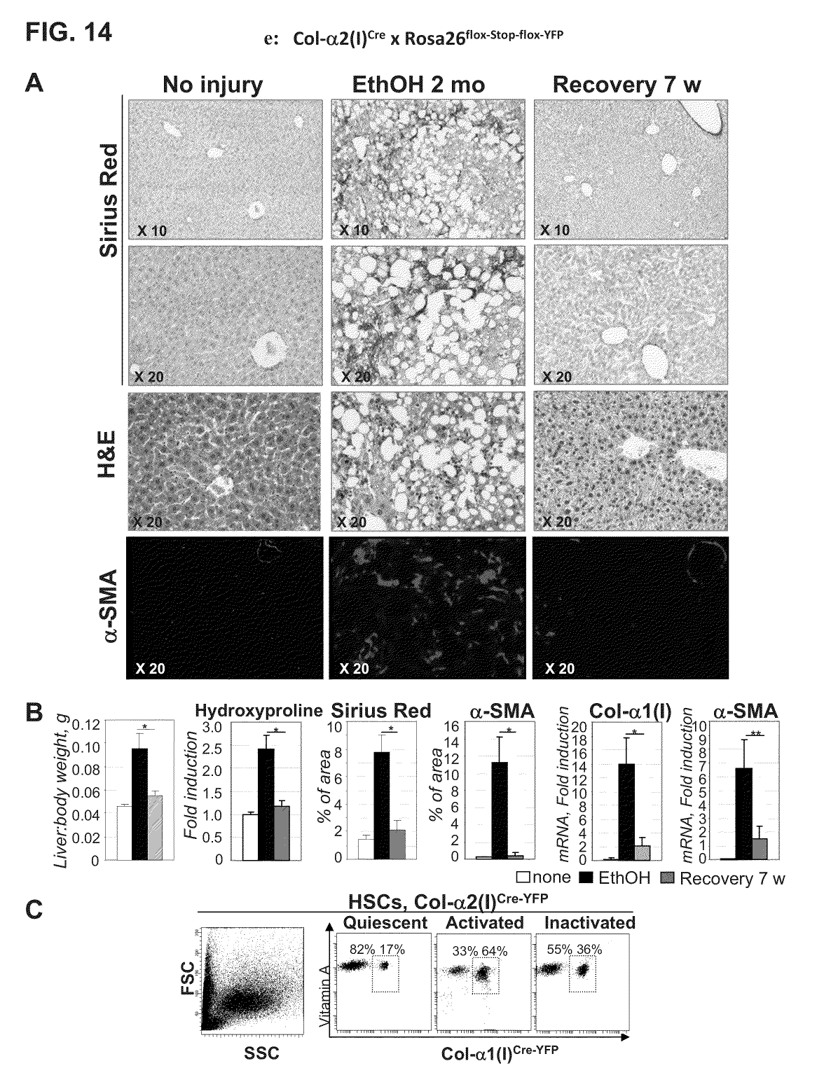

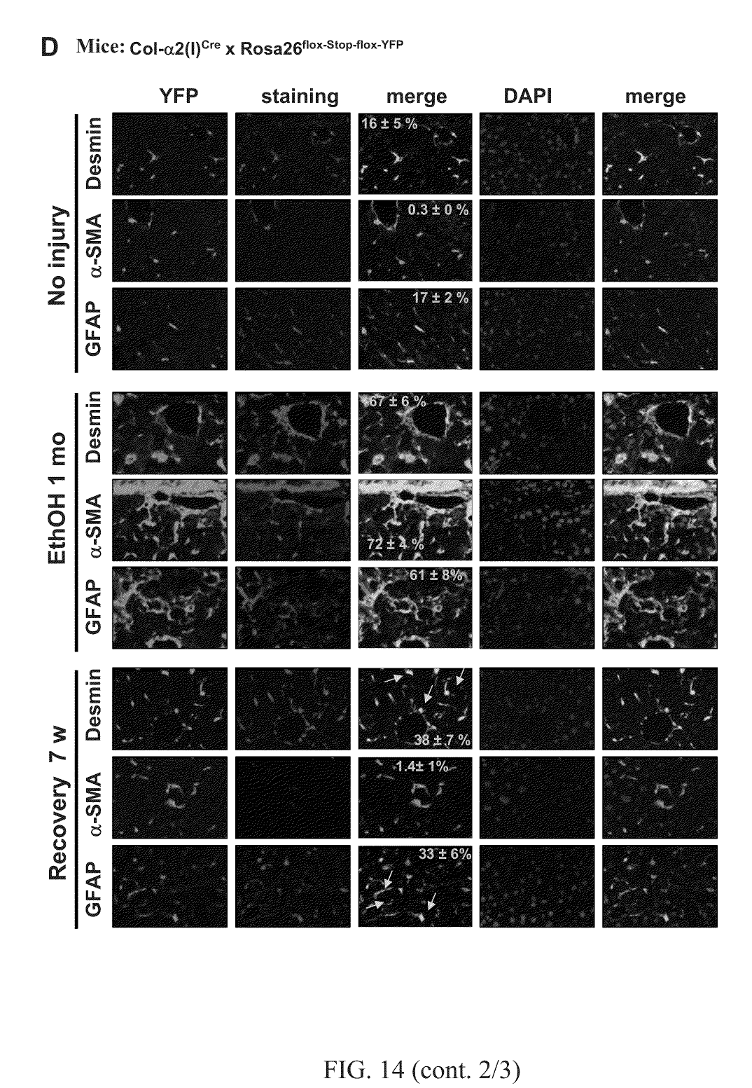

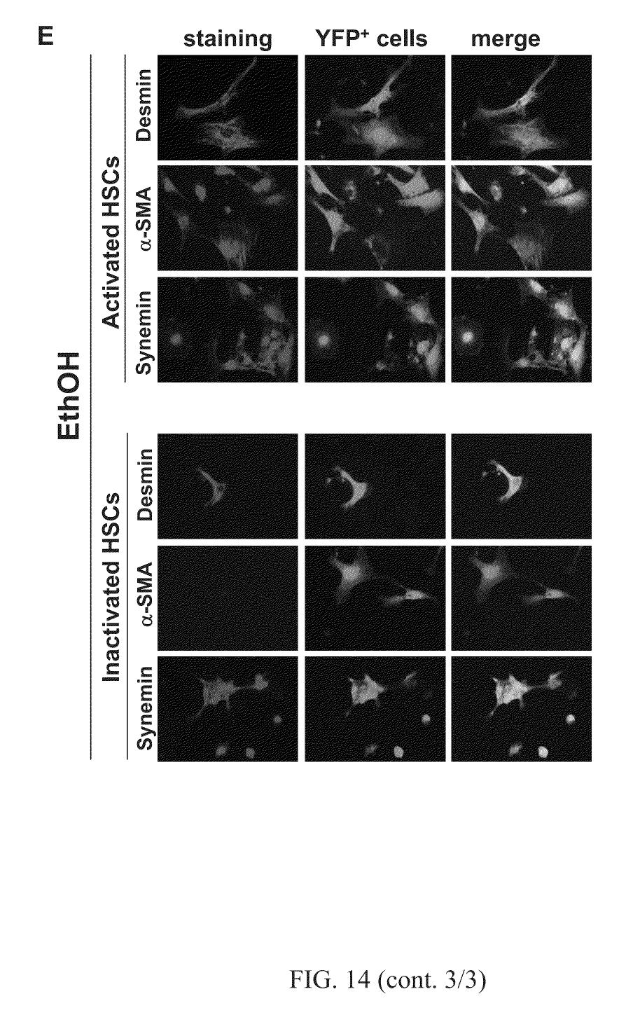

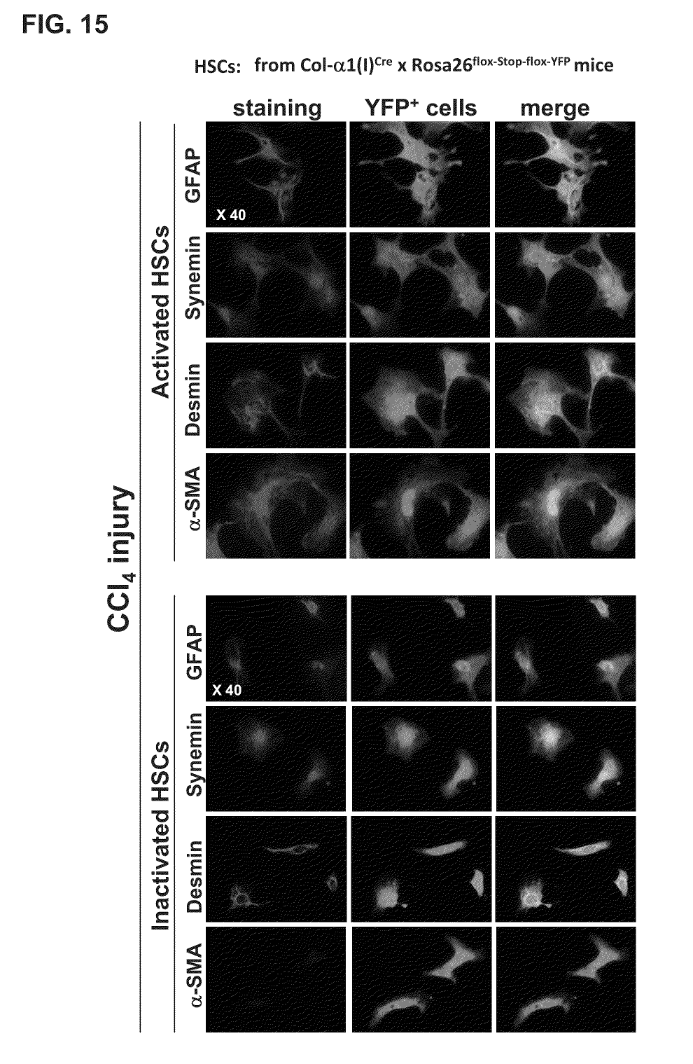

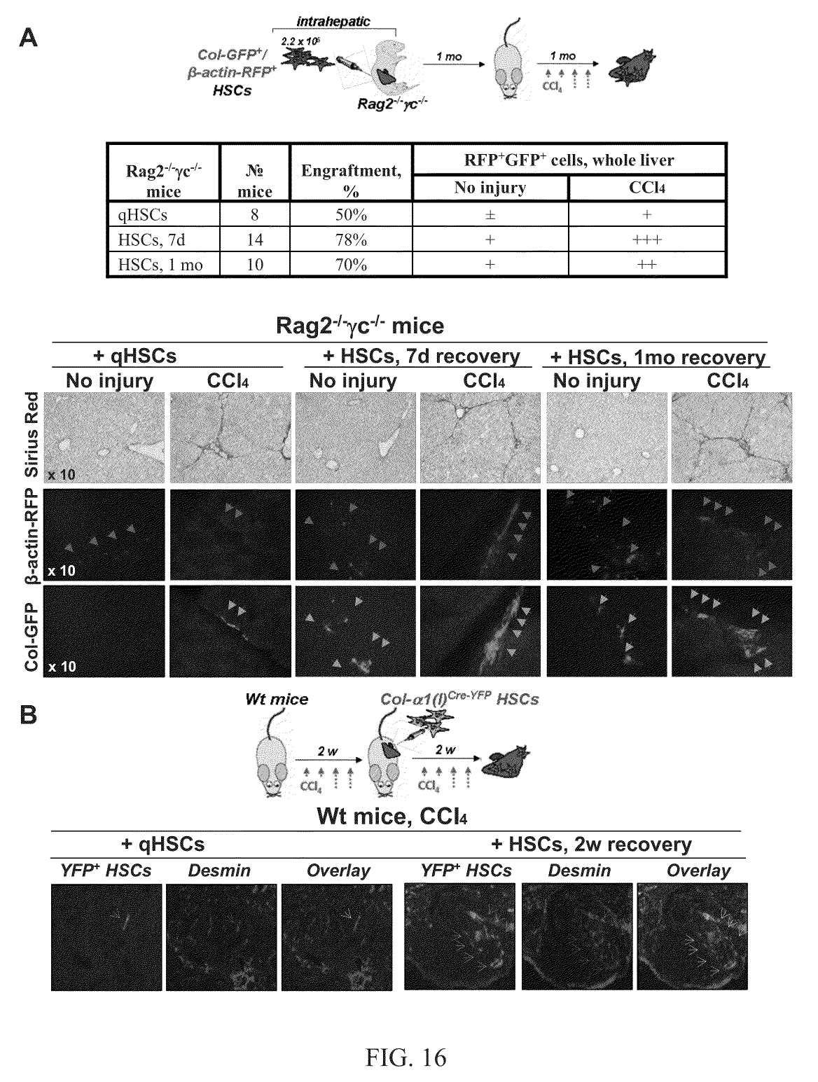

It was determined if survival of aHSCs/myofibroblasts occurs during regression of alcohol induced liver fibrosis. Liver fibrosis (and steatosis) was induced in Col-α2(I)^{Cre-YFP} mice (Collagen-α2(I)^{Cre}×Rosa26^{flox-Stop-flox-YFP} mice) by intragastric alcohol feeding for 2 months (FIG. 14A-B). Liver fibrosis (and steatosis) regressed in these mice 7 weeks after withdrawal from ethanol feeding. Flow cytometry demonstrated that genetic labeling (YFP⁺) was achieved in 64±5% of myofibroblasts, and persisted in 36±4% of Vitamin A⁺ YFP⁺ HSCs upon recovery from fibrosis (FIG. 14C). These findings were confirmed by immunohistochemistry (FIG. 14D-E). YFP expression persisted in 38±7% of Desmin⁺ HSCs/myofibroblasts following regression of liver fibrosis after withdrawal from ethanol, despite the disappearance of myofibroblasts (α-SMA expressed in 1.4±1% of YFP⁺ HSCs/myofibroblasts, FIG. 14D). Thus, two models of regression of liver fibrosis demonstrate survival of iHSCs.

### Example 9

### iHSCs Demonstrate an Increased Response to Repeated Fibrogenic Stimuli

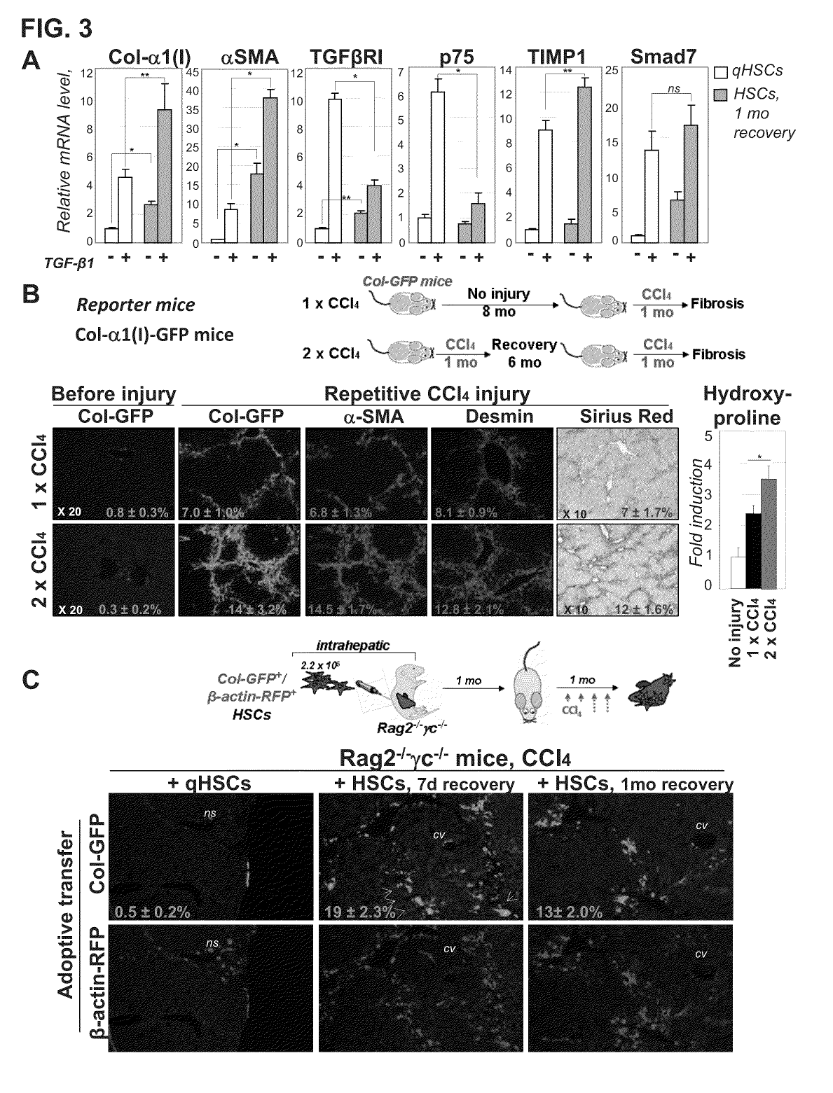

Purified iHSCs had a similar phenotype as qHSCs (Desmin⁺, GFAP⁺, Synemin⁺, α-SMA⁻, FIGS. 14E & 15). However, expression of myofibroblast-specific genes (Col-α1(I), α-SMA, TIMP-1) was induced more strongly in cultured TGF-β1-treated iHSCs than in qHSCs (FIG. 3A). In concordance, Col-GFP mice subjected to two rounds of CCl₄ injury separated by a 6-month interval to allow complete recovery (2×CCl₄) developed more severe fibrosis than littermates treated with one round of CCl₄ (1×CCl₄, FIG. 3B). Thus, culture and in vivo data indicated that iHSCs with a history of activation are more effectively activated than qHSCs.

### Example 10

### Adoptively Transferred HSCs (1 mo Recovery), but not qHSCs, Contribute to Liver Fibrosis in Mice

To test this hypothesis, HSCs were isolated from Col-GFP⁺/β-actin-RFP⁺ mice that were uninjured or after recovery from CCl₄-induced fibrosis (7 days, 1 month), and adoptively transferred into livers of the newborn Rag2^{-/-} γc^{-/-} mice(11) (FIG. 3C). One month later, these Rag2^{-/-}γc^{-/-} mice were subjected to CCl₄-injury, and fibrotic livers were analyzed for the presence of GFP⁺RFP⁺ HSCs. Highest engraftment (70-78%) was achieved in mice transplanted with HSCs after 7 d or 1 mo recovery (versus qHSCs, 50%; FIG. 16A). Unlike qHSCs, which were mostly scattered under the capsule or in liver parenchyma and constituted only 0.5±0.2% of total HSCs, HSCs from the recovering livers were incorporated into the myofibroblast population in recipient mice, and contributed 19±2.3% and 13±2.0% of total HSCs, respectively (FIG. 3C). Moreover, despite poor engraftment, comparable results were observed in CCl₄-treated wild type mice adoptively transferred with qHSCs or HSCs (2 w recovery) from Col-α1(I)^{Cre-YFP} mice (FIG. 16B). Taken together, iHSCs are primed to differentiate into myofibroblasts more rapidly in response to recurrent stimuli.

### Example 11

### Inactivated HSCs Gradually Down Regulate Collagen-α1(I)

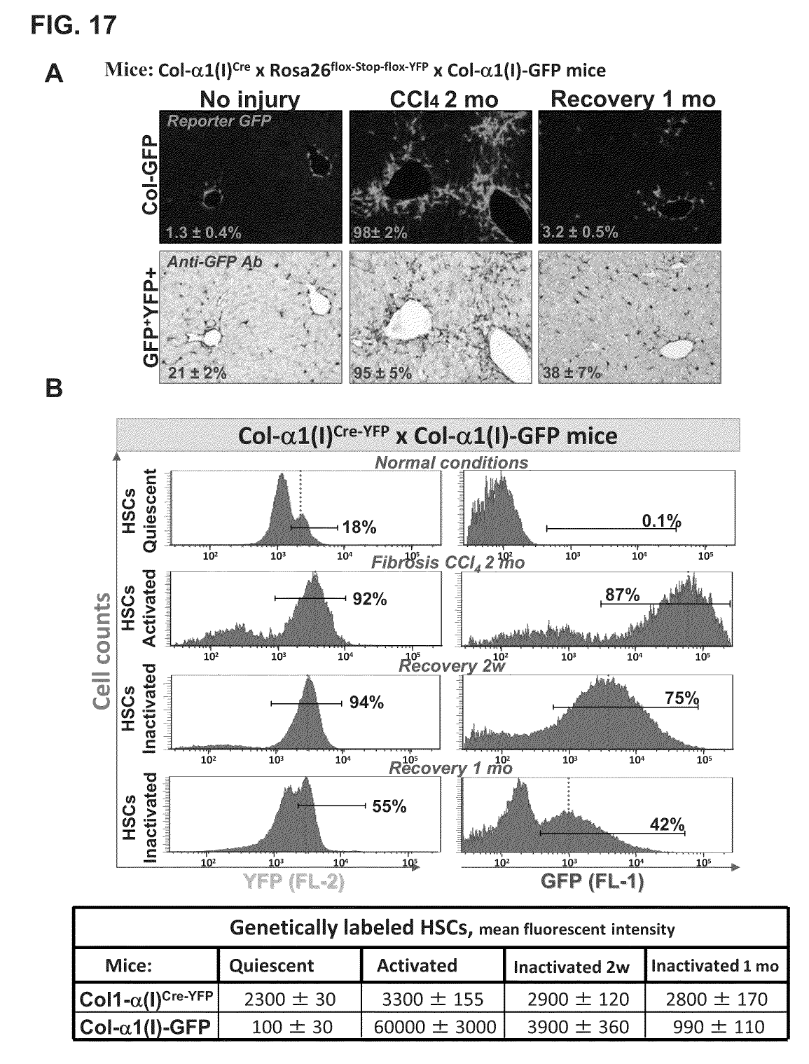

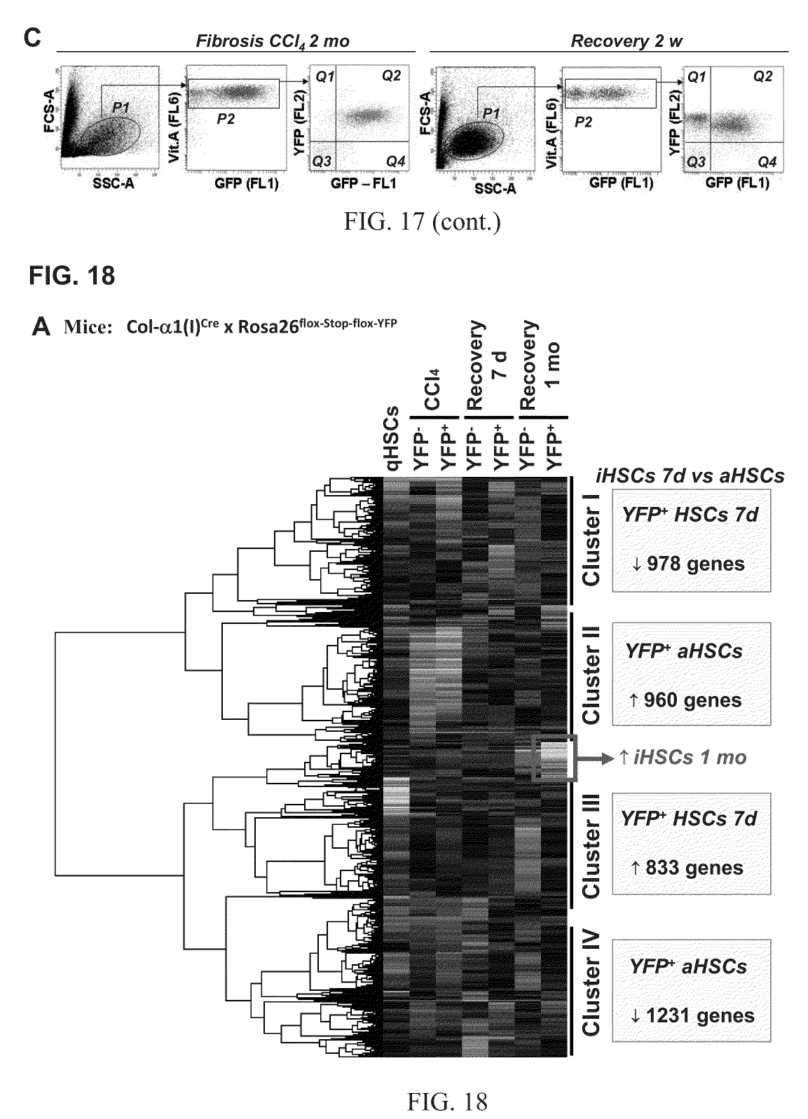

To further characterize iHSCs, Col-α1(1)^{Cre-YFP} mice were crossed with Col-GFP mice, and genetically labeled HSCs (YFP⁺) were analyzed for expression of collagen-α1(I) in real time (GFP⁺, FIG. 17). Following CCl₄ treatment (2 mo, FIG. 17A), all YFP HSCs expressed GFP. After 1 mo. recovery from fibrosis, YFP HSCs had decreased GFP expression. Similar results were obtained by flow cytometry (FIG. 17B-C), which allowed simultaneous detection of Vitamin A, YFP and GFP expression(12) in isolated HSCs. As expected, qHSCs lacked GFP expression and HSCs expressed GFP in response to CCl₄ (87±5%, FIG. 17B). Following a 2 week recovery from CCl₄, decreased GFP expression was observed in 75±3% of HSCs, of which 92±4% still expressed YFP. The mean fluorescent intensity (mfi) of GFP expression was strongly reduced in YFP HSCs at this time (≈4×10³ mfi, compared to aHSCs ≈6×10⁴ mfi; FIG. 17B). GFP expression (≈1×10³ mfi) decreased further in 42±4% of HSCs after 1 mo recovery and correlated with the number of YFP iHSCs (55±3%). Thus, inactivation of HSCs occurs gradually and steadily during recovery from CCl₄-induced fibrosis. Interestingly, 45% of HSCs after 1 mo. recovery had no history of collagen expression (YFP⁻), and represent new qHSCs (FIG. 17B).

### Example 12

### iHSCs Acquire a New Phenotype Distinct from qHSCs

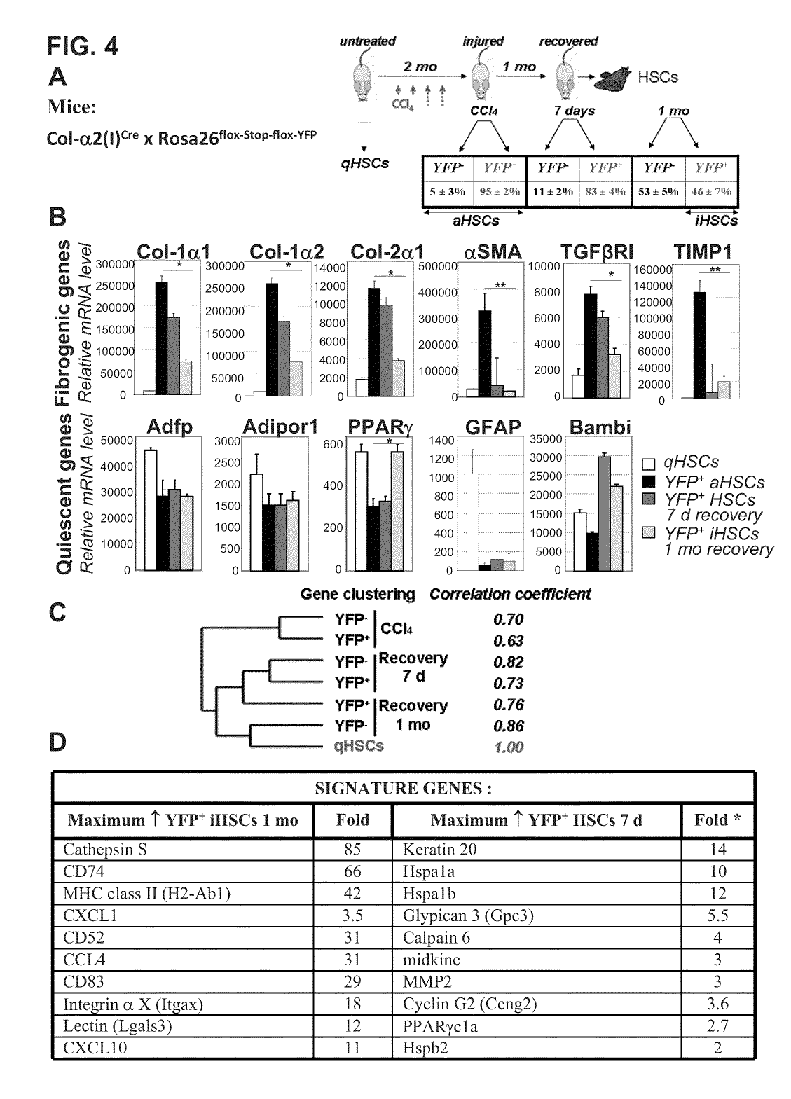

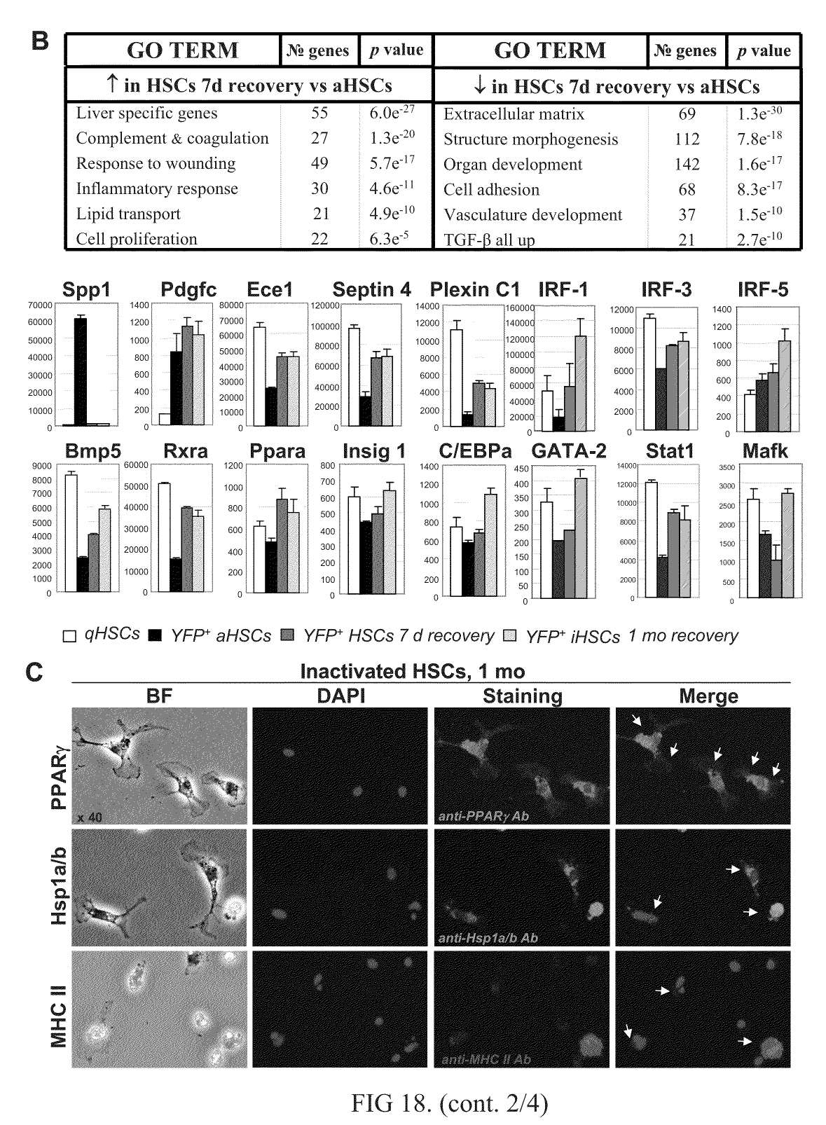

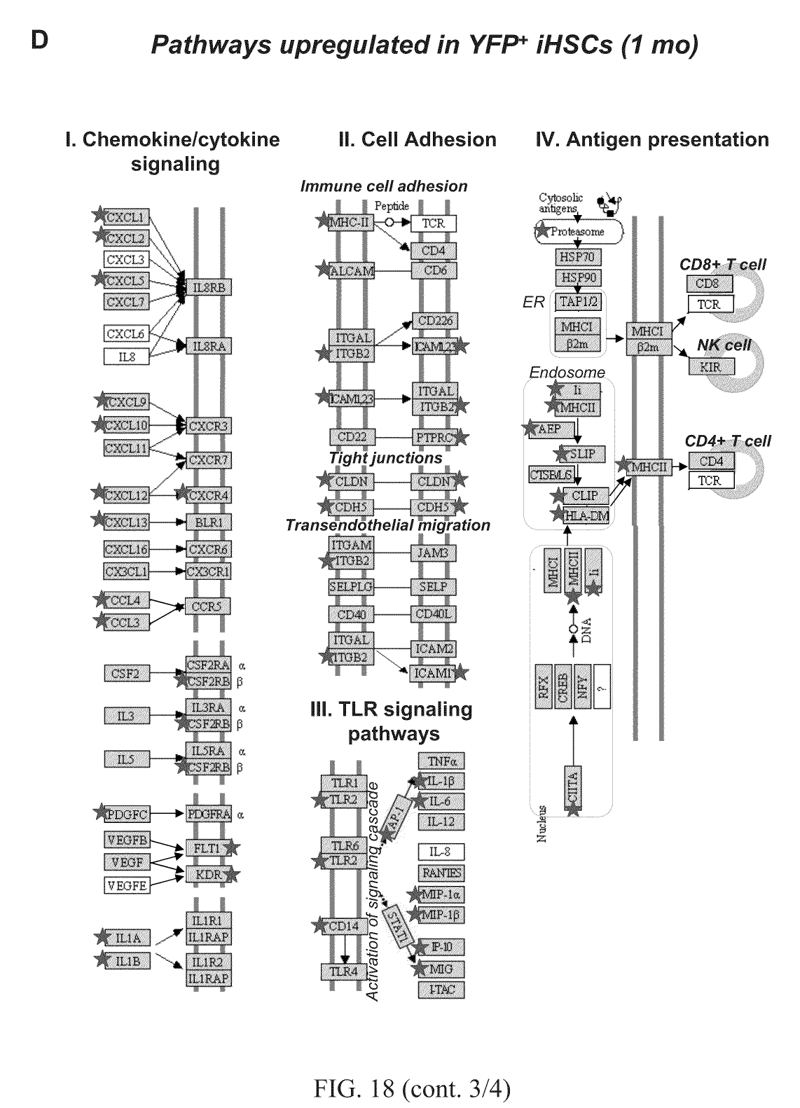

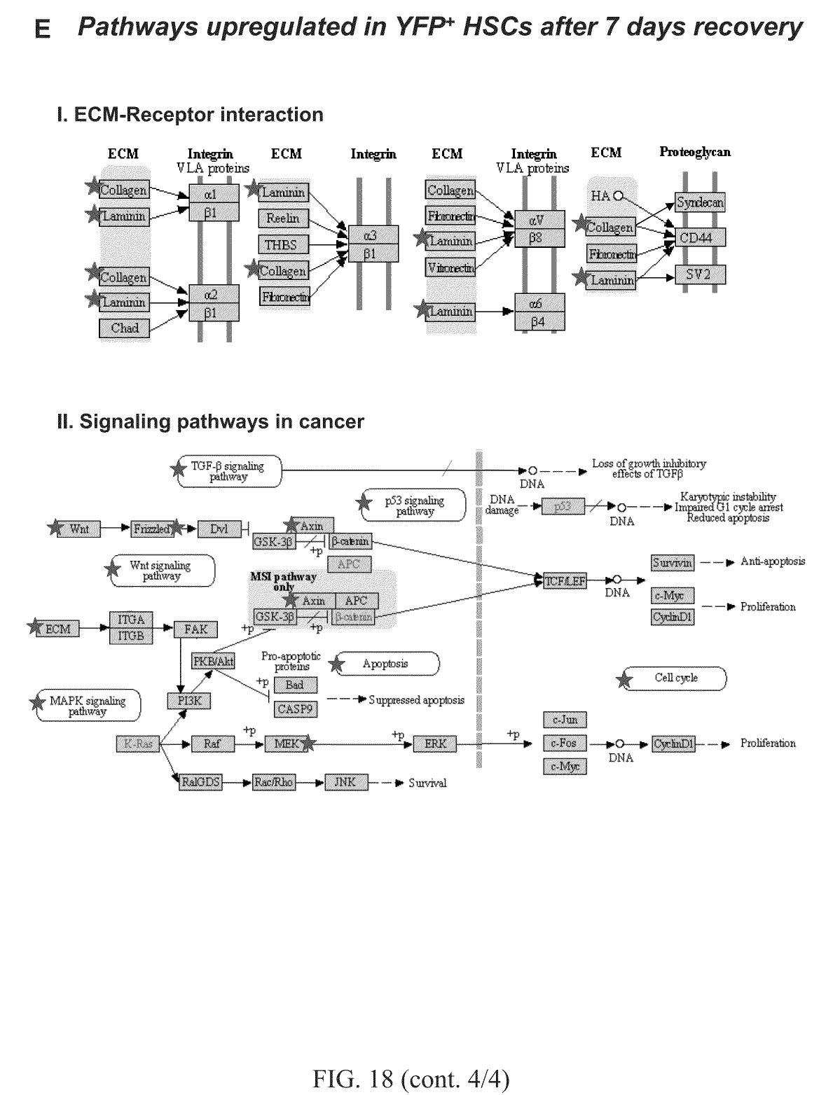

To assess changes in global gene expression, inactivated YFP⁺ HSCs (iHSCs, 1 mo recovery) were evaluated by the whole mouse genome microarray and compared to qHSCs, aHSCs and HSCs after 7-days recovery (FIG. 4A). We confirmed that YFP⁺ iHSCs downregulated fibrogenic genes (Col-1α1, Col-1α2, Col-1α1, α-SMA, TGFβRI and TIMP1) during recovery from fibrosis, but failed to obtain a quiescent phenotype (upregulated PPARγ and Bambi, but not other quiescence-associated genes Adfp, Adipor1, or GFAP(5), FIG. 4B). Unsupervised clustering of gene expression profiles revealed that YFP⁺ iHSCs (1 mo.) exhibit an intermediate profile between that of qHSCs and YFP⁺ HSCs (7 days recovery), but share more similarity to qHSCs than aHSCs (FIG. 4C-D). Similar results were obtained using correlation coefficient analysis comparing expression profiles to qHSCs (FIG. 4C), and unsupervised clustering of gene-specific expression profiles (FIG. 18A-C).

### Example 13

### Activation of Hspa1a/b May Promote Survival of iHSCs at day 7 of Recovery from Liver Fibrosis

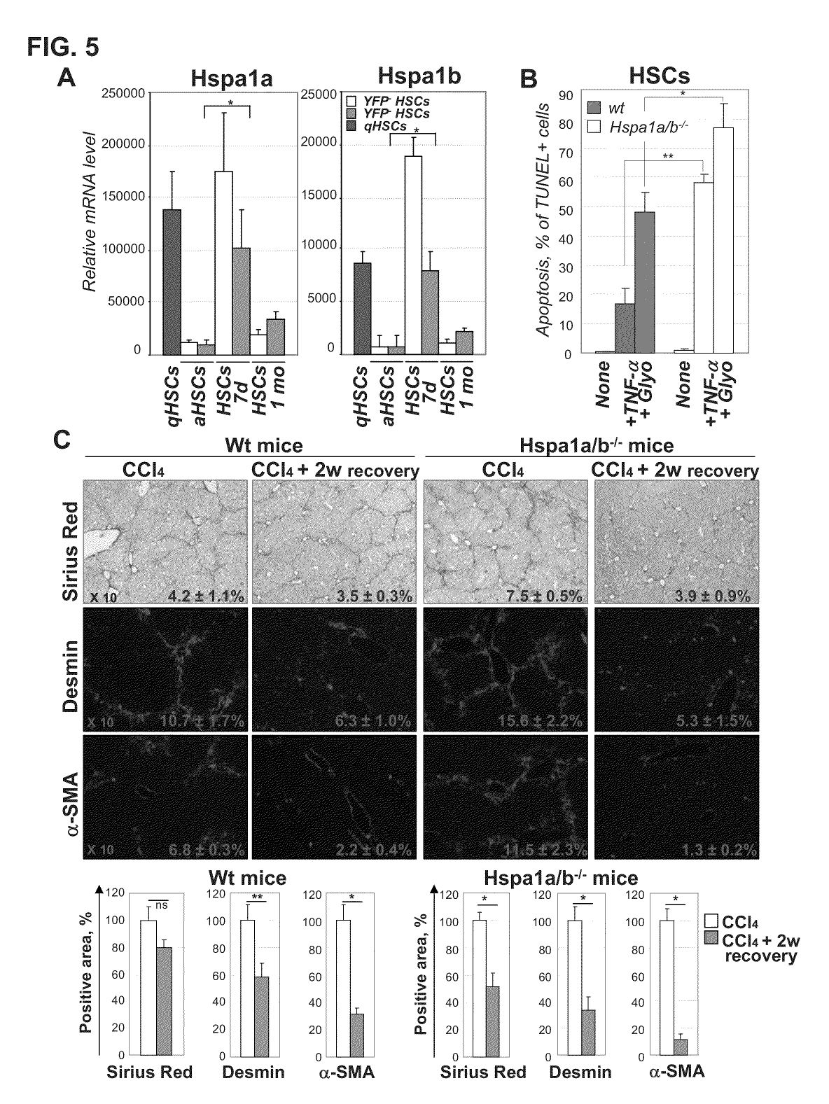

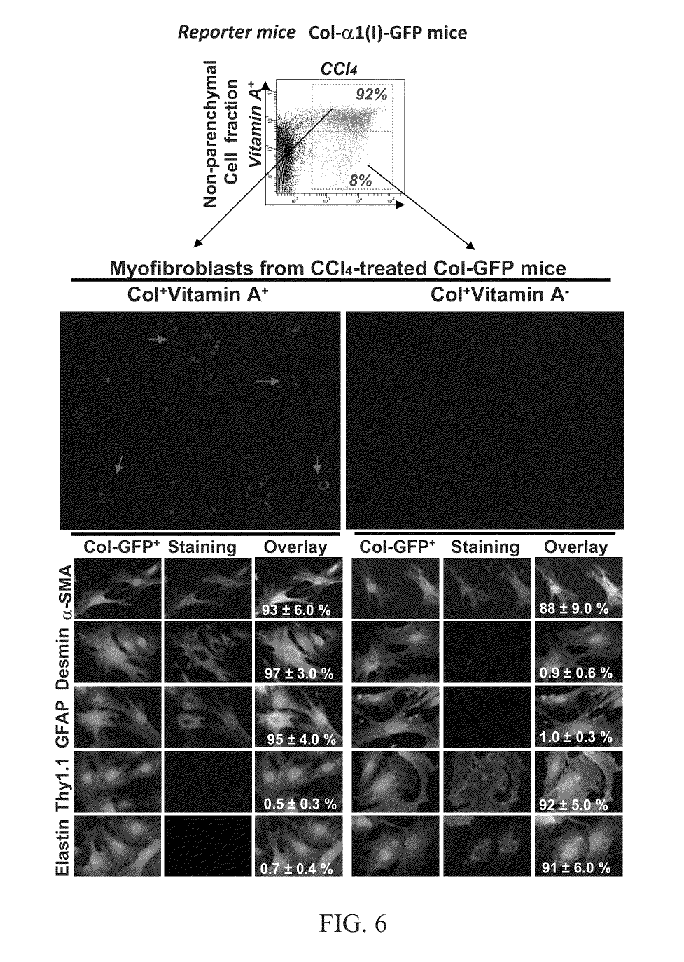

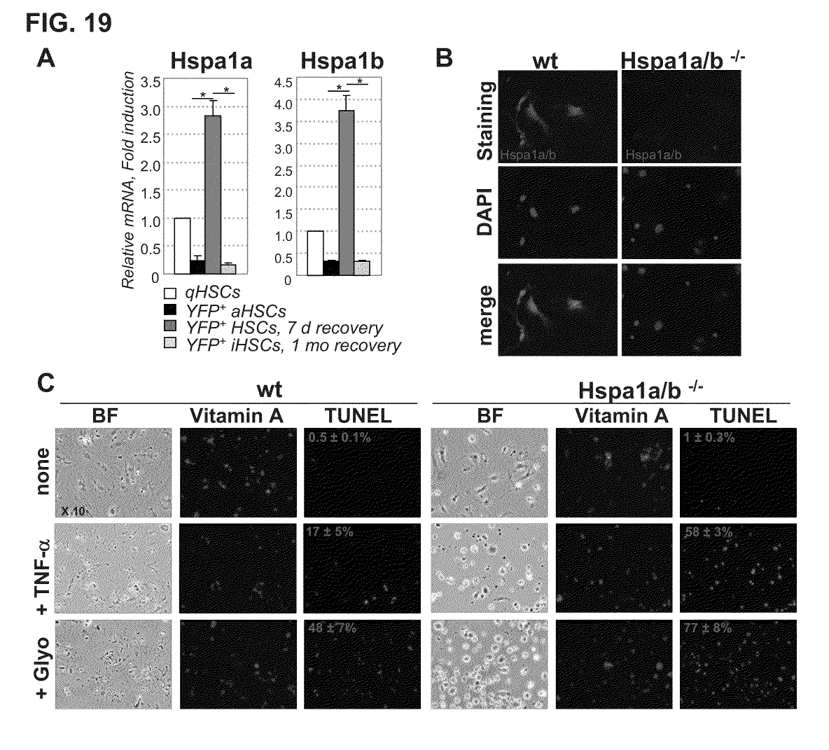

To understand how YFP⁺ iHSCs escape apoptosis, we examined the signaling pathways in YFP⁺ HSCs after 7 days recovery (FIG. 18B, 18E). In particular, expression of the anti-apoptotic Hspa1a/b genes was strongly but transiently induced these HSCs (FIG. 5A, FIG. 19A, to the levels comparable to qHSCs), but was dramatically downregulated in aHSCs and HSCs after 1 mo recovery (FIG. 5A, FIG. 19A).

We examined if Hspa1a/b would impact survival of cultured HSCs. For this purpose, HSCs were isolated from CCl₄-treated Hspa1a/b^{-/-} and wild type mice (FIG. 19B), and cultured 5 days on plastic. Hspa1a/b^{-/-} HSCs had a rounded shape and exhibited growth retardation (cell number ratio ko:wt-1:1.7, FIG. 19C). Moreover, Hspa1a/b^{-/-} HSCs were more susceptible to glyotoxin-(13) and TNF-α-induced apoptosis(14) (FIG. 5B and FIG. 19C). Therefore, upregulation of Hspa1a/b genes may promote survival of iHSCs during recovery from fibrosis.

### Example 14

### Resolution of CCl₄-Induced Fibrosis is Expedited in Hspa1a/b^{-/-} Mice

It was hypothesized that the loss of survival signals in Hspa1a/b^{-/-} HSCs would result in increased clearance of aHSCs after recovery from CCl₄-induced fibrosis. To test this, Hspa1a/b^{-/-} and wild type mice were subjected to CCl₄-induced liver injury. As expected, Hspa1a/b^{-/-} mice developed more severe fibrosis (probably due to increased hepatocyte death)(15) than the wild type littermates (FIG. 5C). However, after stopping CCl₄ treatment, regression of liver fibrosis was strongly accelerated in Hspa1a/b^{-/-} mice compared to wild type mice (decreased 49% vs. 20% by Sirius red staining respectively). Hspa1a/b^{-/-} livers also had a greater loss of α-SMA⁺Desmin⁺ aHSCs compared to wild type mice (decreased 68% vs. 40% of Desmin⁺ positive area, respectively, FIG. 5C). Thus, Hspa1a/b is required so that iHSCs persist in the recovering liver.

### Example 15

### Expression of Mesothelin is Upregulated in PFs in Response to Injury

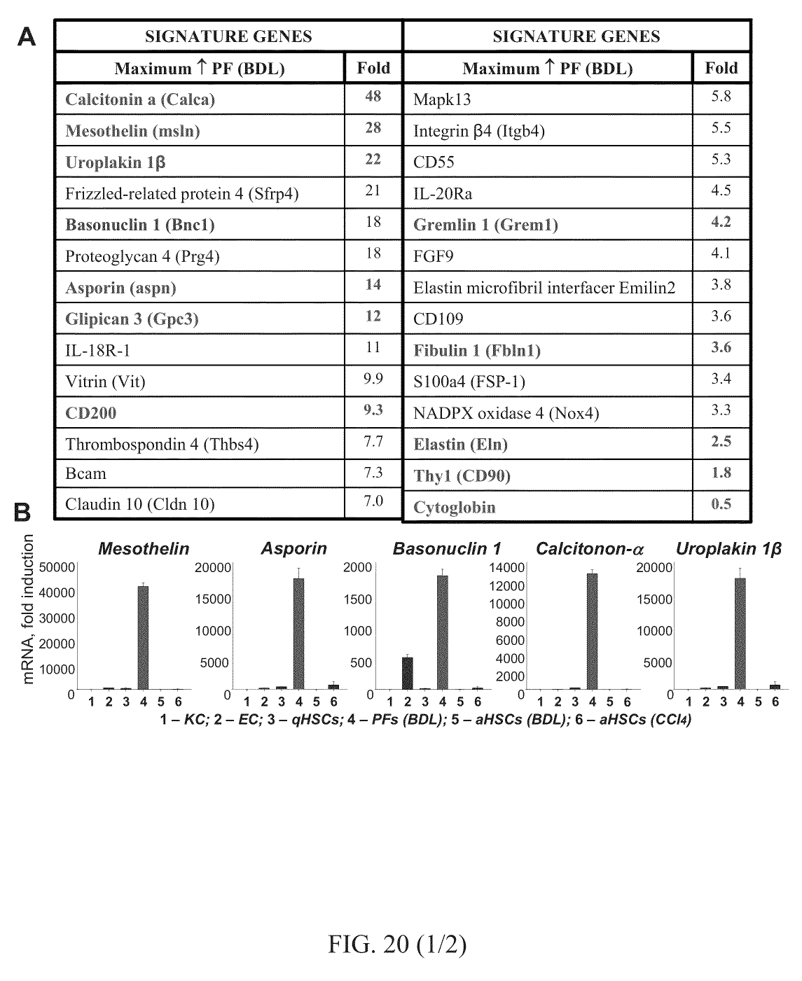

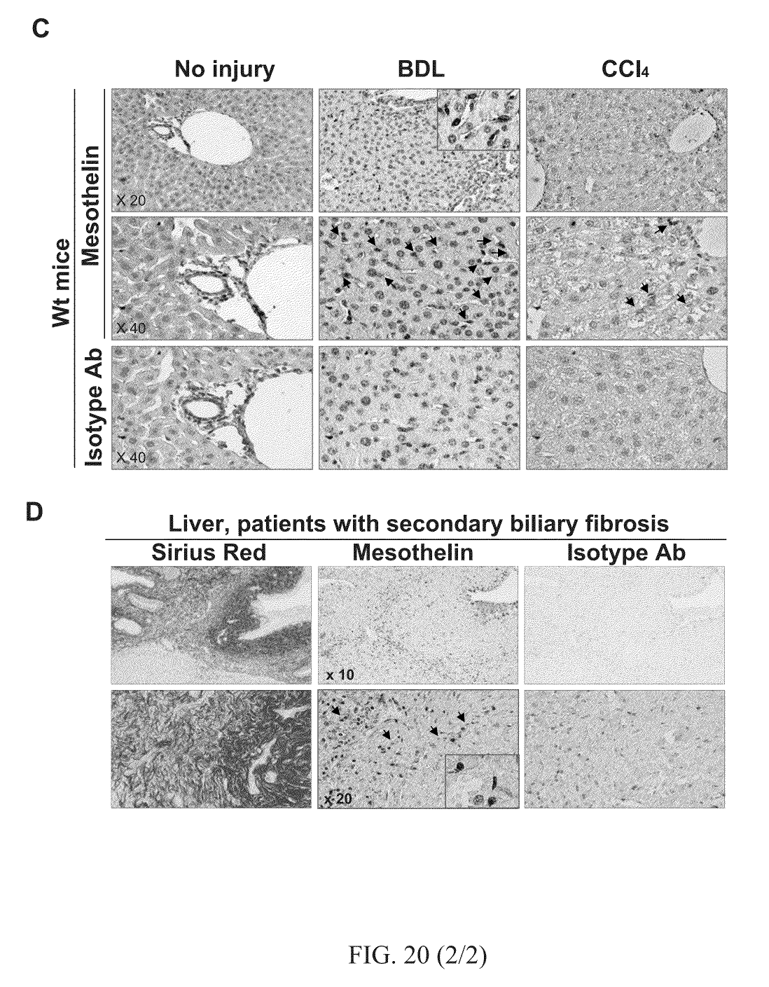

Expression level of selected genes was compared in aPF, aHSCs and other cell types in the liver, and confirmed specific upregulation of asporin, basonuclin 1, calcitonon-α, uroplakin 1β in aPFs (FIG. 20A-D). Hence, expression of mesothelin was detected only in isolated PF, and was clearly absent in aHSCs, KC, EC, and most closely correlated with the expression level of mesothelin in the whole liver. Expression of mesothelin was then examined in BDL- and CCl₄-induced liver tissues. Very few mesothelin⁺ cells were identified in livers of CCl₄-treated mice. In contrast, mesothelin was widely expressed in livers of BDL-treated mice, and showed expression pattern similar to Thy1 and elastin. Moreover, expression of mesothelin was detected in fibrotic lesions in patients with secondary biliary fibrosis. These data indicate that mesothelin may serve as a marker of PFs.

### Discussion of Results

Clinical and experimental hepatic fibrosis regresses dramatically with removal of the underlying etiological agent. Myofibroblasts are aSMA⁺ Collagen Type I⁺ cells that are absent from the normal uninjured liver, rapidly emerge in fibrotic liver to produce the fibrous scar, and completely disappear with regression of liver fibrosis(1, 2). In hepatotoxic-induced liver fibrosis (such as CCl₄ or intragastric alcohol feeding), quiescent hepatic stellate cells (GFAP⁺Desmin⁺SMA⁻Col⁻ qHSCs) undergo activation to become the major source of myofibroblasts (GFAP⁺Desmin⁺aSMA⁺Col⁺ aHSCs). The above results demonstrate the use of genetic markers to address the composition of fibrogenic myofibroblasts. The fate (determined using irreversible genetic labeling using Cre-lox system) of these aHSCs/myofibroblasts during regression of liver fibrosis is determined using transgenic mice specifically generated for this study, Collagen-Cre mice. The data demonstrate that aHSCs/myofibroblasts are cleared by two mechanisms: 1) As previously reported, some myofibroblasts undergo cell death by apoptosis(2); and 2) Some myofibroblasts revert to a previously unrecognized inactive phenotype (iHSCs) that is similar to, but distinct from, quiescent HSCs.

Reversal of fibrosis is associated with increased collagenase activity, activation of macrophages/Kupffer cells secreting matrix metalloproteinases, and matrix degradation (1). Senescence and apoptosis of activated HSCs plays a significant role in resolution of liver fibrosis by eliminating the cell type responsible for producing the fibrotic scar (2, 3). Here. the data demonstrate that some aHSCs undergo apoptosis, while other aHSCs escapes apoptosis, lose expression of fibrogenic genes, and persist in the liver in an inactivated phenotype. This phenomenon was demonstrated using two models of liver fibrosis with different etiologies: CCl₄ and alcohol-induced liver injury. These data suggest that inactivation of aHSCs/myofibroblasts is a common feature of regression of liver fibrosis.

Studies in culture suggest that aHSCs can revert to a quiescent phenotype, associated with expression of lipogenic genes (Adfp, Adipor1, CREBP, PPAR-γ)(5) and storage of vitamin A in lipid droplets. Depletion of peroxisome proliferator-activated receptor gamma (PPAR-γ) constitutes a key molecular event for HSC activation, and ectopic over-expression of this nuclear receptor results in the phenotypic reversal of activated HSC to quiescent cells in culture(5). The treatment of activated HSCs with an adipocyte differentiation cocktail, over-expression of SREBP-1c, or culturing on basement membrane-like ECM(16) results in up-regulation of adipogenic transcription factors and causes morphologic and biochemical reversal of activated HSCs to quiescent cells(17). Applicants in vivo cell fate mapping studies demonstrate that iHSCs survive apoptosis during reversal of liver fibrosis with a new phenotype that is similar to, but distinct from, the original qHSCs.

The data presented herein confirms that HSCs transiently express collagen Type I during development (E16.5-P14), but do not spontaneously become myofibroblasts. This observation explains the presence of genetically labeled qHSCs with a history of collagen expression in livers of uninjured adult mice. These genetically labeled qHSCs possess a quiescent phenotype, indistinguishable from qHSCs with no history of collagen expression. In addition, transient activation of HSCs is required for liver regeneration following partial hepatectomy(18), but the subsequent fate of these HSCs is currently unknown. In turn, after 1 month of regression from CCl₄-induced liver fibrosis, aHSCs/myofibroblasts do not fully revert to a quiescent phenotype. iHSCs downregulate the fibrogenic genes Collagen-α1(1), Collagen-α1(2), α-SMA, TGFβRI and TIMP1, upregulate some quiescence associated genes (PPARγ and Bambi) to levels comparable to qHSCs, but did not re-acquire high expression of GFAP, Adfp and Adipor1 (5). These genetically labeled iHSCs constituted ≈50% of total HSCs in the liver 1 mo after reversal of liver fibrosis. Interestingly, the remaining HSCs have no history of activation, highly resemble qHSCs phenotypically, and represent new qHSCs generated from residual YFP⁻ qHSCs or from a precursor cell population. Although during development HSCs originate from submesothelial mesenchymal cells(19), the source of HSC replenishment is unknown. Using bone marrow chimeric mice, several studies have indicated that HSCs originate from endogenous liver cells and not from a bone marrow derived progenitor cell (8).

Unlike aHSCs, iHSCs completely downregulate expression of fibrogenic genes, but in response to TGFβ1, more rapidly activate into myofibroblasts than qHSCs. Consistent with the concept of iHSCs being more fibrogenic than qHSCs, a previously injured and recovered liver develops more fibrosis than a naïve liver. Applicants directly tested this concept in vivo by adoptive transfer of HSCs into livers of immunodeficient Rag2^{-/-}γc^{-/-} mice. Unlike previous ectopic transfer experiments (20, 21), HSCs (1 mo. recovery) were transplanted into their natural liver environment, and their response to CCl₄-injury was monitored. Here, Applicants demonstrated that iHSCs activate and fully integrate into the fibrous scar in recipient mice more efficiently than qHSCs. Thus, in culture and in vivo iHSCs are activated more effectively than naïve qHSCs, so that the previously injured liver generates more fibrous scar in response to a repeated injury.

It is not clear why some aHSCs escape apoptosis and inactivate, while other HSCs die after cessation of the injury. Applicants' data suggest that survival of iHSCs requires the upregulation of pro-survival signals, such as induction of heat shock proteins(22). Two members of Hsp70 family of heat shock proteins, Hspa1a and Hspa1b(22), that play a protective role against stress-induced apoptosis(23), were strongly and transiently upregulated in HSCs after 7 days of reversal of fibrosis (when apoptosis of other aHSCs is highest) compared with the aHSCs in fibrotic liver. Furthermore, Applicants demonstrated that genetic ablation of Hspa1a/b renders aHSCs more susceptible to TNF-α(14) and glyotoxin-induced(24) apoptosis in culture. In concordance, regression of liver fibrosis was strongly accelerated in Hspa1a/b^{-/-} mice, and was associated with increased disappearance of α-SMA⁺Desmin⁺ HSCs. These data suggest that Hspa1a/b regulate HSC survival, while PPAR-γ drives HSC inactivation during reversal from liver fibrosis.

Characterization of New Markers of aPFs.

In an attempt to distinguish aPFs from aHSCs and other myofibroblasts, the “signature genes” characteristic for aPFs were identified. Applicants confirmed that aPFs do not express cytoglobin, a marker of aHSCs. Applicants also confirmed that mRNA expression of Thy1, elastin, Gremlin 1 and Fibulin1 clearly discriminates between aPFs and aHSCs. In turn, expression of cofilin-1 distinguished aPFs from CCl₄-activated HSCs, but not from BDL-activated aHSCs. Furthermore, unique expression of calcitonin α (fold induction ↑48), mesothelin (↑28), uroplakin 1β (↑22), basonuclin 1 (↑18), asporin (↑14), glipican 3 (↑12), CD200 (↑9.3) mRNA in PFs identifies these genes as potential new markers of activated PFs. Upregulation of these genes specifically in aPFs was confirmed by RT-PCR.

### EQUIVALENTS

Those skilled in the art will recognize, or be able to ascertain using no more than routine experimentation, many equivalents to the specific embodiments of the invention described herein. Such equivalents are intended to be encompassed by the following claims.

### LITERATURE CITED

1. Bataller R & Brenner DA (2005) Liver fibrosis. J Clin Invest 115(2):209-218.
2. Iredale JP, et al. (1998) Mechanisms of spontaneous resolution of rat liver fibrosis. Hepatic stellate cell apoptosis and reduced hepatic expression of metalloproteinase inhibitors. J Clin Invest 102(3):538-549.
3. Krizhanovsky V, et al. (2008) Senescence of activated stellate cells limits liver fibrosis. Cell 134(4):657-667.
4. Friedman S L, Roll F J, Boyles J, & Bissell D M (1985) Hepatic lipocytes: the principal collagen-producing cells of normal rat liver. Proc Natl Acad Sci USA 82(24):8681-8685.

5. She H, Xiong S, Hazra S, & Tsukamoto H (2005) Adipogenic transcriptional regulation of hepatic stellate cells. J Biol Chem 280(6):4959-4967.
6. Lee CG, et al. (2004) Early growth response gene 1-mediated apoptosis is essential for transforming growth factor beta1-induced pulmonary fibrosis. J Exp Med 200(3):377-389.
7. Huby AC, et al. (2009) Restoration of podocyte structure and improvement of chronic renal disease in transgenic mice overexpressing renin. PLoS One 4(8):e6721.
8. Seki E, et al. (2007) TLR4 enhances TGF-beta signaling and hepatic fibrosis. Nat Med 13(11):1324-1332.
9. Lee K S, Buck M, Houglum K, & Chojkier M (1995) Activation of hepatic stellate cells by TGF alpha and collagen type I is mediated by oxidative stress through c-myb expression. J Clin Invest 96(5):2461-2468.
10. Stefanovic B & Brenner DA (2003) 5′ stem-loop of collagen alpha 1(1) mRNA inhibits translation in vitro but is required for triple helical collagen synthesis in vivo. J Biol Chem 278(2):927-933.
11. Goldman JP, et al. (1998) Enhanced human cell engraftment in mice deficient in RAG2 and the common cytokine receptor gamma chain. British journal of haematology 103(2):335-342.
12. Hawley T S, Herbert D J, Eaker S S, & Hawley R G (2004) Multiparameter flow cytometry of fluorescent protein reporters. Methods Mol Biol 263:219-238.
13. Wright M C, et al. (2001) Gliotoxin Stimulates the Apoptosis of Human and Rat Hepatic Stellate Cells and Enhances the Resolution of Liver Fibrosis in Rats. Gastroenterology 121(3):685-698.
14. Siegmund S V, et al. (2007) The endocannabinoid 2-arachidonoyl glycerol induces death of hepatic stellate cells via mitochondrial reactive oxygen species. FASEB J 21(11):2798-2806.
15. Ikeyama S, Kusumoto K, Miyake H, Rokutan K, & Tashiro S (2001) A non-toxic heat shock protein 70 inducer, geranylgeranylacetone, suppresses apoptosis of cultured rat hepatocytes caused by hydrogen peroxide and ethanol. J Hepatol 35(1):53-61.
16. Wells RG (2008) The role of matrix stiffness in regulating cell behavior. Hepatology 47(4):1394-1400.
17. Tsukamoto H (2005) Adipogenic phenotype of hepatic stellate cells. Alcohol Clin Exp Res 29(11 Suppl):132S-133S.
18. Kalinichenko VV, et al. (2003) Foxf1 +/− mice exhibit defective stellate cell activation and abnormal liver regeneration following CCl4 injury. Hepatology 37(1):107-117.
19. Asahina K, et al. (2009) Mesenchymal origin of hepatic stellate cells, submesothelial cells, and perivascular mesenchymal cells during mouse liver development. Hepatology 49(3):998-1011.
20. Yin Z, Wu W, Fung J J, Lu L, & Qian S (2007) Cotransplanted hepatic stellate cells enhance vascularization of islet allografts. Microsurgery 27(4):324-327.
21. Winau F, et al. (2007) Ito cells are liver-resident antigen-presenting cells for activating T cell responses. Immunity 26(1):117-129.
22. Yenari M A, et al. (2005) Antiapoptotic and anti-inflammatory mechanisms of heat-shock protein protection. Ann N Y Acad Sci 1053:74-83.
23. Gabai V L, et al. (2000) Hsp72-mediated suppression of c-Jun N-terminal kinase is implicated in development of tolerance to caspase-independent cell death. Mol Cell Biol 20(18):6826-6836.
24. Wright MC, et al. (2001) Gliotoxin stimulates the apoptosis of human and rat hepatic stellate cells and enhances the resolution of liver fibrosis in rats. Gastroenterology 121(3):685-698.
25. Yata Y, et al. (2003) DNase I-hypersensitive sites enhance alpha1(I) collagen gene expression in hepatic stellate cells. Hepatology 37(2):267-276.
26. Florin L, et al. (2004) Cre recombinase-mediated gene targeting of mesenchymal cells. Genesis 38(3):139-144.
27. Tsukamoto H, Mkrtchyan H, & Dynnyk A (2008) Intragastric ethanol infusion model in rodents. Methods Mol Biol 447:33-48.
28. Xiong S, et al. (2008) Hepatic macrophage iron aggravates experimental alcoholic steatohepatitis. Am J Physiol Gastrointest Liver Physiol 295(3):G512-521.
29. Yan A W, et al. (Enteric dysbiosis associated with a mouse model of alcoholic liver disease. Hepatology.
30. de Hoon M J, Imoto S, Nolan J, & Miyano S (2004) Open source clustering software. Bioinformatics 20(9):1453-1454.
31. Saldanha A J (2004) Java Treeview—extensible visualization of microarray data. Bioinformatics 20(17):3246-3248.
32. Dennis G, Jr., et al. (2003) DAVID: Database for Annotation, Visualization, and Integrated Discovery. Genome Biol 4(5):P3.
33. Eyden B. The myofibroblast: phenotypic characterization as a prerequisite to understanding its functions in translational medicine. J Cell Mol Med 2008; 12:22-37.
34. Majno G, Gabbiani G, Hirschel B J, et al. Contraction of granulation tissue in vitro: similarity to smooth muscle. Science 1971; 173:548-50.
35. Gabbiani G, Ryan G B, Majne G. Presence of modified fibroblasts in granulation tissue and their possible role in wound contraction. Experientia 1971; 27:549-50.
36. Schurch W, Seemayer T A, Gabbiani G. The myofibroblast: a quarter century after its discovery. Am J Surg Pathol 1998; 22:141-7.
37. Kisseleva T, Brenner D A. Mechanisms of fibrogenesis. Exp Biol Med (Maywood) 2008; 233:109-22.
38. Parola M, Marra F, Pinzani M. Myofibroblast-like cells and liver fibrogenesis: Emerging concepts in a rapidly moving scenario. Mol Aspects Med 2008; 29:58-66.
39. Iredale JP. Models of liver fibrosis: exploring the dynamic nature of inflammation and repair in a solid organ. J Clin Invest 2007; 117:539-48.
40. Geerts A. History, heterogeneity, developmental biology, and functions of quiescent hepatic stellate cells. Semin Liver Dis 2001; 21:311-35.
41. Sachs B D, Baillie G S, McCall J R, et al. p75 neurotrophin receptor regulates tissue fibrosis through inhibition of plasminogen activation via a PDE4/cAMP/PKA pathway. J Cell Biol 2007; 177:1119-32.
42. Kendall T J, Hennedige S, Aucott R L, et al. p75 Neurotrophin receptor signaling regulates hepatic myofibroblast proliferation and apoptosis in recovery from rodent liver fibrosis. Hepatology 2009; 49:901-10.
43. Senoo H, Kojima N, Sato M. Vitamin a-storing cells (stellate cells). Vitam Horm 2007; 75:131-59.
44. Bataller R, Schwabe R F, Choi Y H, et al. NADPH oxidase signal transduces angiotensin II in hepatic stellate cells and is critical in hepatic fibrosis. J Clin Invest 2003; 112:1383-94.
45. Winau F, Hegasy G, Weiskirchen R, et al. Ito cells are liver-resident antigen-presenting cells for activating T cell responses. Immunity 2007; 26:117-29.
46. Fallowfield J A, Mizuno M, Kendall T J, et al. Scar-associated macrophages are a major source of hepatic matrix metalloproteinase-13 and facilitate the resolution of murine hepatic fibrosis. J Immunol 2007; 178:5288-95.
47. Kisseleva T, Brenner DA. Hepatic stellate cells and the reversal of fibrosis. J Gastroenterol Hepatol 2006; 21 Suppl 3:S84-7.
48. Kisseleva T, Uchinami H, Feirt N, et al. Bone marrow-derived fibrocytes participate in pathogenesis of liver fibrosis. J Hepatol 2006; 45:429-38.
49. Higashiyama R, Moro T, Nakao S, et al. Negligible Contribution of Bone Marrow-Derived Cells to Collagen Production During Hepatic Fibrogenesis in Mice. Gastroenterology 2009.
50. Tuchweber B, Desmouliere A, Bochaton-Piallat ML, et al. Proliferation and phenotypic modulation of portal fibroblasts in the early stages of cholestatic fibrosis in the rat. Lab Invest 1996; 74:265-78.
51. Guyot C, Lepreux S, Combe C, et al. Hepatic fibrosis and cirrhosis: the (myo)fibroblastic cell subpopulations involved. Int J Biochem Cell Biol 2006; 38:135-51.
52. Kinnman N, Francoz C, Barbu V, et al. The myofibroblastic conversion of peribiliary fibrogenic cells distinct from hepatic stellate cells is stimulated by platelet-derived growth factor during liver fibrogenesis. Lab Invest 2003; 83:163-73.
53. Dudas J, Mansuroglu T, Batusic D, et al. Thy-1 is an in vivo and in vitro marker of liver myofibroblasts. Cell Tissue Res 2007; 329:503-14.
54. Yovchev M I, Zhang J, Neufeld DS, et al. Thymus cell antigen-1-expressing cells in the oval cell compartment. Hepatology 2009; 50:601-11.
55. Wells R G, Kruglov E, Dranoff J A. Autocrine release of TGF-beta by portal fibroblasts regulates cell growth. FEBS Lett 2004; 559:107-10.
56. Knittel T, Kobold D, Saile B, et al. Rat liver myofibroblasts and hepatic stellate cells: different cell populations of the fibroblast lineage with fibrogenic potential. Gastroenterology 1999; 117:1205-21.

All publications, patent applications, patents, and other references mentioned herein are incorporated by reference in their entireties.

We claim:

1. A method for reducing one or more symptoms of fibrosis in a subject, comprising administering to said subject a therapeutic amount of one or more compounds that upregulate one or more of Hspa1a/b gene, PPARα, PPARγ, HSP70, HSP40, Hyaluronan synthase 1, GATA2, C/EBPa, BMPS, septin 4, Bambi, cathepsin S and H, neural proteins: synaptogyrin 1, synaptotagmin XIII, GFAP, transcription factors: Spi-C transcription factor (spi/PU.1 related), Spi-B transcription factor (spi-1/PU.related), PU.1-IRF, IRF-1 and 3 and 5, ISRE, Stat1, Pax5, Mafk2, ISGF3-g1; BL34 regulator of G-protein signaling 1, or Rnd1-Rho family GTPase, in an activated fibrogenic myofibroblast cell or fibrogenic myofibroblast-like cell in an amount sufficient to decrease or inhibit the fibrosis.

2. The method of claim 1 wherein the one or more compounds upregulate PPARγ, PPARα and/or Hspa1a/b genes.

3. The method of claim 1, wherein the fibrotic condition is a fibrotic condition of the lung, liver, heart, kidney, skin, gastrointestinal tract or a combination thereof.

4. The method according to claim 3, wherein the fibrotic condition of the liver is chosen from fatty liver disease, steatohepatitis, primary and secondary biliary cirrhosis, cirrhosis, alcohol induced liver fibrosis, biliary duct injury, biliary fibrosis, hepatic fibrosis associated with hepatitis infection, autoimmune hepatitis, non-alcoholic fatty liver disease or progressive massive fibrosis.

5. The method according to claim 1, wherein the compound is selected from one or more of PPARα agonist, PPARγ agonist, Hsp70 upregulator, HSP40 upregulator, Hspa1a/b upregulator, Hyaluronan synthase 1 upregulator or GATA2 upregulator.

6. The method according to claim 5 wherein the PPARα agonist is fenofibrate, WY14643, gemfibrozil, or ciprofibrate.

7. The method according to claim 5, wherein the PPARγ agonist is thiazolidinediones, or 15-deoxy-delta (12, 14)-prostaglandin J2.

8. The method according to claim 5, wherein the HSP70 and HSP40 upregulator is 17-allyamino-demthoxygeldanamycin.

9. The method according to claim 5, wherein the Hspa1a/b upregulator is taurolidine or tumor necrosis factor receptor apoptosis inducing ligand.

10. The method according to claim 5, wherein the PPARα agonist, or PPARγ agonist is given in combination with upregulators of HSP70, HSP40, Hspa1a/b, Hyaluronan synthase 1 or GATA2.

11. The method according to claim 1, wherein the compound induces inactivation of fibrogenic myofibroblasts.

12. The method according to claim 11, wherein the fibrogenic myofibroblast is a hepatic stellate cell.

13. The method according to claim 1, wherein the compound or compounds are given in combination with other antifibrotics, corticosteroids, anti-inflammatories, immunosuppressants, chemotherapeutic agents, anti-metabolites, and immunomodulators.

14. The method according to claim 1, wherein the compound or compounds is given in combination with one or more of adefovir dipivoxil, candesartan, colchicine, combined ATG, mycophenolate mofetil, and tacrolimus, combined cyclosporine microemulsion and tacrolimus, elastometry, everolimus, FG-3019, Fuzheng Huayu, G1262570, glycyrrhizin (monoammonium glycyrrhizinate, glycine, L-cysteine monohydrochloride, interferon gamma-1b, irbesartan, losartan, oltipraz, ORAL IMPACT®., peginterferon alfa-2a, combined peginterferon alfa-2a and ribavirin, peginterferon alfa-2b (SCH 54031), combined peginterferon alpha-2b and ribavirin, praziquantel, prazosin, raltegravir, ribavirin (REBETOL®., SCH 18908), ritonavir-boosted protease inhibitor, pentoxyphilline, tacrolimus, tauroursodeoxycholic acid, tocopherol, ursodiol, and warfarin.

15. The method according to claim 1, wherein the compounds or compounds are given in combination with a compound or compounds that downregulate Ssp1 and/or Pdgfc.

16. A method for detecting hepatic stellate cells(HSCs) or portal fibroblasts (PFs) in a sample, comprising determining the presence of a marker, wherein:
a) the marker is at least one HSC marker selected from vitamin A+, Collagen+, Desmin+, GFAP+, CD146+; or
b) the marker is at least one PF marker selected from Vitamin A−, Collagen +, Thy1.1+, and Elastin+, Mesothelin+.

17. The method according to claim 16, wherein the methods utilize flow cytometry.

18. A method for distinguishing portal fibroblasts (PFs) and Hepatic Stellate Cells (HSCs) in a sample, comprising at least one of
a) determining the presence of at least one HSC marker selected from vitamin A+, Collagen+, Desmin+, GFAP+, CD146+, and
b) determining the presence of at least one PF marker selected from Vitamin A−, Collagen +, Thy1.1+, and Elastin+, Mesothelin+.

19. The method according to claim 18, wherein the methods utilize flow cytometry.
